SEQUENCE LISTING (48 sequences)

SEQ ID NO: 1 - protein, 53 aa, Artificial
  AAP
MENLQQPPLLWDLLQWLQAVAHQWQTITKAPTEWVMPQEIGIAIPHGWATESS

SEQ ID NO: 2 - protein, 239 aa, Artificial
  EGFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 3 - protein, 29 aa, Artificial
  Silk
GAGAGAGQGAGAGAGQGAAAGAGAGAGQT

SEQ ID NO: 4 - protein, 54 aa, Artificial
  SilkE
GAGAGQGAGAGAGQGAAAGAGAGAGQGAGAGAGQGAGAGAGQGAAGAGAGAGQT

SEQ ID NO: 5 - protein, 148 aa, Artificial
  Full MLD
PTPVTAIGPPTTAIQEPPSRIVPTPTSPAIAPPTETMAPPVRDPVPGKPTVTIRTRGAIIQTPTLGPIQPTRVSEAGTTVPGQIRPTLTIPGYVEPTAVITPPTTTTKKPRVSTPKPATPSTDSSTTTTRRPTKKPRTPRPVPRVTTK

SEQ ID NO: 6 - protein, 61 aa, Artificial
  Partial MLD
TLTIPGYVEPTAVITPPTTTTKKPRVSTPKPATPSTDSSTTTTRRPTKKPRTPRPVPRVTT

SEQ ID NO: 7 - protein, 56 aa, Artificial
  Collagen
GSSGVRLWATRQAMLGQVHEVPEGWLIFVAEQEELYVRVQNGFRKVQLEARTPLPR

SEQ ID NO: 8 - protein, 33 aa, Artificial
  Tetramer
AEIEQAKKEIAYLIKKAKEEILEEIKKAKQEIA

SEQ ID NO: 9 - protein, 37 aa, Artificial
  NLS/NoLS
KSRRSRRMMASQPSLITLPARFKSSRTRSTSFRTSSA

SEQ ID NO: 10 - protein, 23 aa, Artificial
  Rpp29
RHKRKEKKKKAKGLSARQRRELR

SEQ ID NO: 11 - protein, 31 aa, Artificial
  AP3D1
RRHRQKLEKDKRRKKRKEKEERTKGKKKSKK

SEQ ID NO: 12 - protein, 19 aa, Artificial
  SV40
KRTADGSEFESPKKKRKVE

SEQ ID NO: 13 - protein, 16 aa, Artificial
  HIV Rev
RQARRNRRRRWRERQR

SEQ ID NO: 14 - protein, 58 aa, Artificial
  T/S rich region
KSPVLQRGPATTTTTSATAPPGGILISTDSTATFHHVTGSDSSTTIGDSGPRDSTSNS

SEQ ID NO: 15 - protein, 11 aa, Artificial
  proline rich region of AAV1
PPAPAPGPCPP

SEQ ID NO: 16 - protein, 11 aa, Artificial
  proline rich region of AAV2
PPAPEPGPCPP

SEQ ID NO: 17 - DNA, 25 bp, Artificial
  primer
aacatgctacgcagagagggagtgg

SEQ ID NO: 18 - DNA, 29 bp, Artificial
  primer
catgagacaaggaacccctagtgatggag

SEQ ID NO: 19 - protein, 53 aa, Artificial
  WSN
KRMENLNKKVDDGFLDIWTYNAELLVLLENERTLDFHDLNVKNLYEKVKSQLK

SEQ ID NO: 20 - protein, 37 aa, Artificial
  4BR
STSRSRRSRRRTARQRWLITLPARFRSLRTRRTNCRT

SEQ ID NO: 21 - protein, 64 aa, Artificial
  5BR
STFKSKRSRCRTPPPPSPTTSPPPSKCLRTTTTSCPTSSATGPRDACRPSLRRSLRCRSTVTRR

SEQ ID NO: 22 - protein, 55 aa, Artificial
  9BR
STFRSKRLRTTMESRPSPITLPARSRSSRTQTISSRTCSGRLTRAASRRSQRTFS

SEQ ID NO: 23 - protein, 196 aa, Artificial
  AAP1
LATQSQSPIHNLSENLQQPPLLWDLLQWLQAVAHQWQTITKAPTEWVMPQEIGIAIPHGWATESSPPAPAPGPCPPTITTSTSKSPVLQRGPATTTTTSATAPPGGILISTDSTATFHHVTGSDSSTTIGDSGPRDSTSNSSTSKSRRSRRMMASQPSLITLPARFKSSRTRSTSFRTSSALRTRAASLRSRRTCS

SEQ ID NO: 24 - protein, 204 aa, Artificial
  AAP2
LETQTQYLTPSLSDSHQQPPLVWELIRWLQAVAHQWQTITRAPTEWVIPREIGIAIPHGWATESSPPAPEPGPCPPTTTTSTNKFPANQEPRTTITTLATAPLGGILTSTDSTATFHHVTGKDSSTTTGDSDPRDSTSSSLTFKSKRSRRMTVRRRLPITLPARFRCLLTRSTSSRTSSARRIKDASRRSQQTSSWCHSMDTSP

SEQ ID NO: 25 - protein, 204 aa, Artificial
  AAP3
LETQSQSQTLNLSENHQQPPQVWDLIQWLQAVAHQWQTITRVPMEWVIPQEIGIAIPNGWATESSPPAPEPGPCPLTTTISTSKSPANQELQTTTTTLATAPLGGILTLTDSTATSHHVTGSDSLTTTGDSGPRNSASSSSTSKLEGSRRTMARRLLPITLPARFKCLRTRSISSRTCSGRRTKAVSRRFQRTSSWSLSMDTSP

SEQ ID NO: 26 - protein, 168 aa, Artificial
  AAP4
LEQATDPLRDQLPEPCLMTVRCVQQLAELQSRADKVPMEWVMPRVIGIAIPPGLRATSRPPAPEPGSCPPTTTTSTSDSERACSPTPTTDSPPPGDTLTSTASTATSHHVTGSDSSTTTGACDPKPCGSKSSTSRSRRSRRRTARQRWLITLPARFRSLRTRRTNCRT

SEQ ID NO: 27 - protein, 201 aa, Artificial
  AAP5
LDPADPSSCKSQPNQPQVWELIQCLREVAAHWATITKVPMEWAMPREIGIAIPRGWGTESSPSPPEPGCCPATTTTSTERSKAAPSTEATPTPTLDTAPPGGTLTLTASTATGAPETGKDSSTTTGASDPGPSESKSSTFKSKRSRCRTPPPPSPTTSPPPSKCLRTTTTSCPTSSATGPRDACRPSLRRSLRCRSTVTRR

SEQ ID NO: 28 - protein, 196 aa, Artificial
  AAP6
LATQSQSPTHNLSENLQQPPLLWDLLQWLQAVAHQWQTITKAPTEWVMPQEIGIAIPHGWATESSPPAPEHGPCPPITTTSTSKSPVLQRGPATTTTTSATAPPGGILISTDSTAISHHVTGSDSSTTIGDSGPRDSTSSSSTSKSRRSRRMMASRPSLITLPARFKSSRTRSTSCRTSSALRTRAASLRSRRTCS

SEQ ID NO: 29 - protein, 196 aa, Artificial
  AAP7
LATQSQSPTLNLSENLQQRPLVWDLVQWLQAVAHQWQTITKVPTEWVMPQEIGIAIPHGWATESLPPAPEPGPCPPTTTTSTSKSPVKLQVVPTTTPTSATAPPGGILTLTDSTATSHHVTGSDSSTTTGDSGPRSCGSSSSTSRSRRSRRMTALRPSLITLPARFRYSRTRNTSCRTSSALRTRAACLRSRRTSS

SEQ ID NO: 30 - protein, 197 aa, Artificial
  AAP8
LATQSQFQTLNLSENLQQRPLVWDLIQWLQAVAHQWQTITKAPTEWVVPREIGIAIPHGWATESSPPAPEPGPCPPTTTTSTSKSPTGHREEPPTTTPTSATAPPGGILTLTDSTATFHHVTGSDSSTTTGDSGPRDSASSSSTSRSRRSRRMKAPRPSPITSPAPSRCLRTRSTSCRTFSALPTRAACLRSRRTCS

SEQ ID NO: 31 - protein, 197 aa, Artificial
  AAP9
LATQSQSQTLNQSENLPQPPQVWDLLQWLQVVAHQWQTITKVPMEWVVPREIGIAIPNGWGTESSPPAPEPGPCPPTTITSTSKSPTAHLEDLQMTTPTSATAPPGGILTSTDSTATSHHVTGSDSSTTTGDSGLSDSTSSSSTFRSKRLRTTMESRPSPITLPARSRSSRTQTISSRTCSGRLTRAASRRSQRTFS

SEQ ID NO: 32 - DNA, 591 bp, Artificial
  AAP1
ctggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatga

SEQ ID NO: 33 - DNA, 615 bp, Artificial
  AAP2
ctggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctga

SEQ ID NO: 34 - DNA, 615 bp, Artificial
  AAP3
ctggagactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttagaggggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctga

SEQ ID NO: 35 - DNA, 507 bp, Artificial
  AAP4
ctggagcaggcgacggaccccctgagggatcaacttccggagccatgtctgatgacagtgagatgcgtgcagcagctggcggagctgcagtcgagggcggacaaggtgccgatggagtgggtaatgcctcgggtgattggcattgcgattccacctggtctgagggccacgtcacgaccaccagcaccagaacctgggtcttgcccacctacaacaaccacctctacaagcgactcggagagagcctgcagtccaacacctacaacggattctccaccccctggggatactttgacttcaaccgcttccactgccacttctcaccacgtgactggcagcgactcatcaacaacaactggggcatgcgacccaaagccatgcgggtcaaaatcttcaacatccaggtcaaggaggtcacgacgtcgaacggcgagacaacggtggctaataaccttaccagcacggttcagatctttgcggactcgtcgtacgaactgccgtacgtga

SEQ ID NO: 36 - DNA, 606 bp, Artificial
  AAP5
ctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctga

SEQ ID NO: 37 - DNA, 591 bp, Artificial
  AAP6
ctggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatga

SEQ ID NO: 38 - DNA, 612 bp, Artificial
  AAP7
ctggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattcctcagtacggctacctga

SEQ ID NO: 39 - DNA, 594 bp, Artificial
  AAP8
ctggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatga

SEQ ID NO: 40 - DNA, 594 bp, Artificial
  AAP9
ctggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatga

SEQ ID NO: 41 - protein, 196 aa, Artificial
  AAV1
TGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVF

SEQ ID NO: 42 - protein, 204 aa, Artificial
  AAV2
TGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLT

SEQ ID NO: 43 - protein, 204 aa, Artificial
  AAV3
TGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLT

SEQ ID NO: 44 - protein, 168 aa, Artificial
  AAV4
TGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPY

SEQ ID NO: 45 - protein, 201 aa, Artificial
  AAV5
AGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYAT

SEQ ID NO: 46 - protein, 196 aa, Artificial
  AAV7
TGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVF

SEQ ID NO: 47 - protein, 197 aa, Artificial
  AAV8
TGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVF

SEQ ID NO: 48 - protein, 197 aa, Artificial; identical to SEQ ID NO: 47
  AAV9

(19) United States
(12) Patent Application Publication
(10) Pub. No.: US 2021/0147487 A1
(43) Pub. Date: May 20, 2021

(54) METHODS AND COMPOSITIONS FOR ENGINEERED ASSEMBLY ACTIVATING PROTEINS (EAAPS)

(71) Applicant: The University of North Carolina at Chapel Hill, Chapel Hill, NC (US)

(72) Inventors: Aravind Asokan, Chapel Hill, NC (US); Sven Moller-Tank, White Plains, NY (US); Long Ping Victor Tse, Chapel Hill, NC (US)

(21) Appl. No.: 17/045,097

(22) PCT Filed: Apr. 4, 2019
(86) PCT No.: PCT/US2019/025788

§ 371 Date: Oct. 2, 2020

Related U.S. Application Data
(60) Provisional application No. 62/652,537, filed on Apr. 4, 2018.

(51) Int. Cl. C07K 14/005; C12N 15/86; C12N 7/00; C07K 16/08

(52) U.S. Cl. CPC C07K 14/005; C12N 15/86; C12N 7/00; C07K 16/081; C07K 2319/09; C12N 2750/14122; C12N 2750/14152; C07K 2319/30; C12N 2750/14143

(57) ABSTRACT

The present invention relates to compositions and methods comprising an engineered assembly activating protein (EAAP).

### STATEMENT OF PRIORITY

This application claims the benefit, under 35 U.S.C. § 119(e), of U.S. Provisional Application Ser. No. 62/652,537, filed Apr. 4, 2018, the entire contents of which are incorporated by reference herein.

### STATEMENT OF GOVERNMENT SUPPORT

This invention was made with government funding under Grant No. HL089221 awarded by the National Institutes of Health. The government has certain rights in the invention.

### STATEMENT REGARDING ELECTRONIC FILING OF A SEQUENCE LISTING

A Sequence Listing in ASCII text format, submitted under 37 C.F.R. § 1.821, entitled 5470-840WO_ST25.txt, 49,849 bytes in size, generated on Apr. 3, 2019 and filed via EFS-Web, is provided in lieu of a paper copy. This Sequence Listing is incorporated by reference into the specification for its disclosures.

### FIELD OF THE INVENTION

The present invention relates to compositions and methods comprising an engineered assembly activating protein (EAAP).

### BACKGROUND OF THE INVENTION

Adeno-associated virus (AAV) is a non-enveloped, single stranded DNA virus belonging to the Dependoparvovirus genus within the Parvoviridae family. The AAV capsid consists of 60 capsid monomers of VP1, 2 and 3 at a ratio of 1:1:10 that package a 4.7 kb single-stranded genome. The AAV genome encodes replication (Rep), capsid (Cap) and assembly activating protein (AAP) open reading frames flanked by inverted terminal repeats (ITRs), which are the sole requirements for genome packaging. As such, the majority of the genome can be replaced by exogenous DNA sequences and packaged inside the AAV capsid to create a recombinant vector for DNA delivery both in vitro and in vivo. Unlike other viruses that are replication competent, AAVs are partially defective as they require a helper virus (such as Adenovirus or Herpes Simplex Virus) for replication. The lack of pathogenicity and ease of genome manipulation have enabled extensive evaluation of recombinant AAV vectors as candidates for clinical gene therapy.

AAV encodes a unique protein, assembly activating protein (AAP), which is not found in other autonomous parvoviruses and is required for AAV capsid assembly. AAP is predicted to be a 20-24 kDa protein, with an actual size ranging from 27-34 kDa, which may be due to post-translational modifications. AAP is encoded from a +1 frame within the Cap ORF overlapping the junction between VP2 and VP3. Introduction of a stop codon within AAP without affecting the coding frame of VP2/3 prevents capsid assembly and virus/vector production. While capsid assembly can be restored by providing AAP in trans, overexpression of wildtype AAP does not increase the vector yield. This suggests that AAP is necessary and sufficient for capsid assembly, but is not a limiting factor for vector production. Cellular localization of AAPs overlaps with the location of capsid assembly, supporting a direct role for AAP. Most AAPs show strong nucleolar localization, while AAP5 and 9 are predominantly nuclear, and excluded from the nucleolus. Although the exact mechanism by which AAP supports AAV assembly is still elusive, several studies have convincingly shown that AAP is important for intracellular capsid expression and localization.

For instance, the steady state level of capsid is dramatically reduced in the absence of AAP. Such regulation must occur at the translational or post-translational level as Cap mRNA expression remains the same regardless of the presence or absence of AAP. Additionally, the AAV2 VPs have been shown to change their cellular localization from cytoplasmic/nuclear to nucleolar in the presence of AAP2. A previous study has shown that the N-terminal region of AAP might interact with the C-terminus of the VP, albeit weakly. Interaction with AAP2 also appears to alter VP conformation as supported by the lack of binding to several conformational specific antibodies. All of the above observations supports the notion that AAP2 acts as a chaperone to stabilize and translocate VP to the site of assembly. However, it should be noted that significant differences in cellular localization and cross-complementation have been reported.

AAPs encoded by different serotypes are closely related, with sequence identity ranging from 48% (AAV4) to 82% (AAV7) relative to AAP1. Phylogenetic analysis of AAP shows the same relationships as AAV VP, where the serotypes 4, 5, 11 and 12 are distinct from the others. The phylogenetic distance is also reflected in the biology of AAP4, -5, -11 and -12 which are unable to complement capsid assembly of other AAV serotypes. Furthermore, in the absence of AAP, AAV4, -5 and -11 are capable of producing 20-40% of AAV particles compared to the WT level; these have been termed “AAP-independent.” At the secondary structure level, using previously defined nomenclature, AAP can be separated into multiple functional regions, N to C terminal: the hydrophobic region (HR), conserved core (CC), proline rich region (PRR), threonine/serine rich region (T/S) and basic region (BR).

### BRIEF DESCRIPTION OF THE DRAWINGS

FIGS. 1A-1D show threonine/serine region (T/S) can be deleted or replaced by heterologous sequences without compromising AAP functions. (1A) Schematic of different AAP mutants. Truncated linker 1-4 (TL1-4) has different internal regions deleted. For others, the T/S was replaced with Bombyx mori silk heavy chain repeats (Silk and SilkE1) or the entire (Full MLD) or partial (Partial MLD) mucin-like domain from murine alpha dystroglycan mucin like domain. All AAP1 constructs have a C9 epitope tag at the C-terminus for immunodetection. (1B) Representative western blot image of VPs, AAPs and actin expression level in HEK293 cells transfected by quadruple transfection for rAAV production after 3 days post-transfection. (1C) Relative vector yield of different AAP mutants normalized to wild type AAP1 (AAP1). (1D) Transduction of rAAV1 packaging luciferase produced from different AAP1 mutants on HEK293 cells at 10,000 vg/cell. Relative light units (RLUs) were normalized to AAP1. Error bars represent 1 standard deviation (1 S.D.) from at least 3 independent experiments. The data were analyzed by a one-tailed Student t-test comparing to AAP1 (*, p<0.05, **, p<0.01, ***, p<0.005).

FIGS. 2A-2F show BR is not the determinant for serotype specificity of AAV capsid assembly. (2A) Schematic of different AAP mutants. The T/S of AAP1 was replaced by EGFP (AAP1E) The basic region (BR) was replaced with that of other serotypes, including AAP4 (4BR), AAP5 (5BR) and AAP9 (9BR). Representative western blot image of (2B) VPs and (2C) AAPs expression as described above in FIG. 1B. (2D) Confocal microscopy of different AAP1E derivatives. AAP1E derivatives (pCDNA3.1-AAPs) are transfected as described herein and visualized by their native EGFP fluorescence, AAP1 was immunostained with α-C9 antibody, and nucleoli by C23 antibody. Nuclei were stained with DAPI. (2E) Relative vector yield using different AAP constructs normalized to wildtype AAP1 (AAP1). (2F) Transduction of rAAV1 packaging luciferase produced from different AAP1 constructs as described above. Relative light units (RLUs) were normalized to AAP1. Error bars and statistical analysis were as described above.

FIGS. 3A-3D show the C-terminal basic region (BR) is important for AAP1 function but can be replaced by heterologous nucleolus localization signal (NoLS). (3A) Schematic of different AAP mutants. The T/S of AAP1 was replaced by EGFP (AAP1E). The BR was replaced by Ribonuclease P subunit 29 NoLS (Rpp29), Adaptor protein in AP-3 complex NoLS (AP3D1), viral derived SV40 NLS (SV40) and HIV Rev NoLS domain (HIV Rev). (3B) Representative western blot image of VPs and AAPs expression as described above in FIG. 1B. (3C) Relative vector yield using different AAP constructs normalized to wildtype AAP1 (AAP1). (3D) Transduction of rAAV1 packaging luciferase produced from different AAP1 constructs as described above. Relative light units (RLUs) were normalized to AAP1. Error bars and statistical analysis were as described above.

FIG. 4 shows confocal microscopy of different AAP1E derivatives. AAP1E derivatives (pCDNA3.1-AAPs) are transfected as described herein. AAP1E derivatives were visualized by their native EGFP fluorescence, nucleoli were immunostained with C23 antibody and nuclei were stained with DAPI.

FIGS. 5A-5D show domain deletion analysis of the N-terminus of AAP1, which reveals that the hydrophobic region (HR) and conserved core (CC) are important for AAP functions. (5A) Schematic of different AAP mutants. The N-terminal domains are either deleted or replaced with their AAP5 counterparts (purple). All AAP mutants have a C9 epitope tag at the C-terminus. (5B) Representative western blot image of viral proteins, AAPs and actin expression of 293 cells transfected by quadruple transfection for rAAV production after 3 days post-transfection. (5C) Relative vector yield using different AAP constructs normalized to wildtype AAP1 (AAP1). (5D) Transduction of rAAV1 packaging luciferase produced from different AAP1 constructs as described above. Relative light units (RLUs) were normalized to AAP1. Error bars and statistical analysis were as described above.

FIG. 6 shows confocal microscopy of different AAP5E and AAP1E derivatives. AAP5E and AAP1E derivatives are transfected as described herein. AAP5E and AAP1E derivatives were visualized by their native EGFP fluorescence, nucleoli were immunostained with C23 antibody and nuclei were stained with DAPI.

FIGS. 7A-7C show immunoprecipitation of VP by different N-terminus mutants of AAP1. (7A) Schematic of different AAP mutants. The N-terminal domains are either deleted or replaced with the AAP5 counterparts. All AAP constructs have their BR replaced by the human-Fc domain at the C-terminus. Representative western blot image of (7B) immunoprecipitation and (7C) input of VP by AAP1Es mutants. Samples were collected after 3 days post-transfection of 293 cells with pXX680, pXR1ΔAAP1, pCDNA3.1-AAP1E-Fc and pTR-CBA-Luc. Detailed protocols are described herein. AAP1E-Fc was pulled down using magnetic protein G beads and is immunostained with Goat α-human HRP antibody and VP is stained with mouse α-capsid antibody (B1).

FIGS. 8A-8C show replacement of T/S with oligomerization domains. (8A) Schematic of different AAP mutants. 8The T/S was replaced with different oligomerization domains: the collagen trimerization domain (Collagen), a synthetic tetramerization domain (Tetra), or the Influenza A/WSN/33 hemagglutinin coiled-coil domain (WSN). All AAP mutants have a C9 epitope tag at the C-terminus. (8B) Representative western blot image of VPs and AAPs expression as described above in FIG. 1B. (8C) Relative vector yield using different AAP constructs normalized to wildtype AAP1 (AAP1). Error bars and statistical analysis were as described above.

FIGS. 9A-9B show dose dependent increase of vector production by AAP1-Collagen. (9A) Representative western blot image of VP, AAPs and actin expression of 293 cells at different dose of AAP1 and AAP1-Collagen provided in trans after 3 days post-transfection. (9B) Relative vector yield of different dose of AAP1 and AAP1-Collagen normalized to standard AAV1 production (pXR1). Error bars represent 1 standard deviation (1 S.D.) from at least 3 independent experiments. The data were analyzed by two-way ANOVA comparing to pXR1 standard production (*, p<0.05, **, p<0.01, ***, p<0.005).

FIGS. 10A-10B show bioinformatics analysis of AAP and VP conservation and homology modeling of AAP. A multiple sequence alignment of AAP and the corresponding VP amino acids was performed and used for further analysis. (10A) The ratio of AAP conservation to VP conservation was calculated for each amino acid position and plotted along a positional axis. Ratios above one indicate greater conservation in AAP and vice versa. Overlaid are various regions and structural elements of each protein. Functional groups in VP are the VP3 start site (arrow), Beta-strand A, B, D and E (βA, βB, βD, βE) and Loop I (LI). (10B) Homology modeling of AAP1E without BR, detailed modeling parameters are described herein. The amino acid sequence of the essential HR and CC are underlined.

FIG. 11. shows an alignment of AAP1-AAP5 and AAP7-AAP9 sequences (Panel A) and an alignment of AAV1-AAV5 and AAV7-AAV9 sequences (Panel B). AAP1: SEQ ID NO:23; AAP2: SEQ ID NO:24; AAP3: SEQ ID NO:25; AAP4: SEQ ID NO:26; AAP5: SEQ ID NO:27; AAP7: SEQ ID NO:29; AAP5: SEQ ID NO:30; AAP9: SEQ ID NO:31. AAV1: SEQ ID NO:41; AAV2: SEQ ID NO:42; AAV3: SEQ ID NO:43; AAV4: SEQ ID NO:44; AAV5: SEQ ID NO:45; AAV7: SEQ ID NO:46; AAV8: SEQ ID NO:47; AAV9: SEQ ID NO:48.

### DETAILED DESCRIPTION OF THE INVENTION

The present invention will now be described with reference to the accompanying drawings, in which representative embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, GenBank accession numbers and other references mentioned herein are incorporated by reference herein in their entirety.

The present invention provides an engineered assembly activating protein (AAP) comprising components: A, B, and C, wherein A can be an N terminal domain having the amino acid sequence MENLQQPPLLWDLLQWLQAVAHQWQTITKAPTEWVMPQEIGIAIPHGWATESS (SEQ ID NO:1); or A can be AAV capsid protein binding domain such as an antibody fragments or binding peptide identified, for example, through phage display; B can be a linker amino acid sequence which can be from about 10 amino acids to about 240 amino acids in length and can comprise, for example: MVSKGEELFTGVVPILVELDGDVNGHKFSVSGEGEGDATYGKLTLKFICTTGKLPVP WPTLVTTLTYGVQCFSRYPDHMKQHDFFKSAMPEGYVQERTIFFKDDGNYKTRAEV KFEGDTLVNRIELKGIDFKEDGNILGHKLEYNYNSHNVYIMADKQKNGIKVNFKIRH NIEDGSVQLADHYQQNTPIGDGPVLLPDNHYLSTQSALSKDPNEKRDHMVLLEFVTA AGITLGMDELYK (EGFP) (SEQ ID NO:2), GAGAGAGQGAGAGAGQGAAAGAGAGAGQT (Silk) (SEQ ID NO:3), GAGAGQGAGAGAGQGAAAGAGAGAGQGAGAGAGQGAGAGAGQGAAGAGAGAG QT (SilkE) (SEQ ID NO:4), PTPVTAIGPPTTAIQEPPSRIVPTPTSPAIAPPTETMAPPVRDPVPGKPTVTIRTRGAIIQT PTLGPIQPTRVSEAGTTVPGQIRPTLTIPGYVEPTAVITPPTTTTKKPRVSTPKPATPSTD SSTTTTRRPTKKPRTPRPVPRVTTK (Full MLD) (SEQ ID NO:5), TLTIPGYVEPTAVITPPTTTTKKPRVSTPKPATPSTDSSTTTTRRPTKKPRTPRPVPRVT T (pMLD) (SEQ ID NO:6), GSSGVRLWATRQAMLGQVHEVPEGWLIFVAEQEELYVRVQNGFRKVQLEARTPLPR (Collagen) (SEQ ID NO:7), and/or AEIEQAKKEIAYLIKKAKEEILEEIKKAKQEIA (Tetramer) (SEQ ID NO:8); or B can comprise a dimerizable domain such as a SpyTag system, an FKBP-based system, a leucine zipper system, an immunoglobulin domain, an intein-based system, a protein domain with secondary structure that can comprise alpha-helical, beta strands, coiled coils, proline helix, beta barrel domains and/or other scaffold domains; or B can comprise a functional domain from other viral or bacterial scaffold proteins that aid in capsid assembly, including, for example, the bacteriophage Protein B or Protein B domain, a phi 29 connector or scaffolding protein, a SPP1 neck protein, or combination thereof; and C can be a C terminal domain having the amino acid sequence KSRRSRRMMASQPSLITLPARFKSSRTRSTSFRTSSA (SEQ ID NO:9); or C can be an exogenous nuclear/nucleolar localization domain (NLS/NoLS), which can be, for example RHKRKEKKKKAKGLSARQRRELR (Rpp29) (SEQ ID NO:10), RRHRQKLEKDKRRKKRKEKEERTKGKKKSKK (AP3D1) (SEQ ID NO:11), KRTADGSEFESPKKKRKVE (SV40) (SEQ ID NO:12), and/or RQARRNRRRRWRERQR (HIV Rev) (SEQ ID NO:13).

In some embodiments, the engineered AAP of this invention, the entire T/S rich region (T/S) having the amino acid sequence KSPVLQRGPATTTTTSATAPPGGILISTDSTATFHHVTGSDSSTTIGDSGPRDSTSNS (SEQ ID NO:14) can be deleted and in some embodiments, the T/S region can be included.

In some embodiments, the engineered AAP of this invention can comprise the existing proline rich region having the amino acid sequence highlighted in the sequences provided herein and identified, respectively, as AAP1 through AAP9. For example, the proline rich region of AAV1 is PPAPAPGPCPP (SEQ ID NO:15); the proline rich region for AAV2 is PPAPEPGPCPP (SEQ ID NO:16), etc., as shown in the amino acid sequences provided herein for the respective AAV serotypes. Thus, it would be apparent to one of skill in the art that AAP1 is the AAP of AAV2; AAP2 is the AAP of AAV2, etc.

In some embodiments, the linker amino acid sequence in the engineered AAP of this invention imparts increased stability, improved ability to support viral capsid assembly, nucleolar transport activity, nuclear transport activity, ability to be detected (e.g., by fluorescence, chemiluminescence), ability to bind other proteins (transcription factors, immune system modulators, cell cycle regulators), ability to bind other nucleic acids (RNA, DNA, PNA), ability to binds other macromolecules (carbohydrates, lipids), ability to form multimers (in the presences or absence of other co-factors), ability to increase virus particle yield (in different production system including mammalian, insect and in vitro assembly), and any combination thereof to the engineered AAP relative to an AAP without the linker amino acid sequence.

In some embodiments, the engineered AAP of any preceding claim, the AAP of this invention is from an adeno-associated virus (AAV), which can be any AAV serotype now known or later identified (e.g., AAV1-10, Rhesus monkey AAV isolates, engineered AAV vectors), including any serotype listed in Table 1.

The present invention further provides a producer cell line for production of AAV particles, comprising a heterologous nucleotide sequence encoding the engineered AAP of this invention.

Nonlimiting examples of a producer cell line of this invention include a mammalian cell line (e.g., HEK293, Hela, Vero, CHO, MDCK), an insect cell line (e.g., Sf9, Sf2), a yeast cell line (e.g., Saccharomyces cerevisiae, Pichia pastoris, and Hansenula polymorpha), a protozoan cell line (Tetrahymena thermophile), or a bacterial cell line (Escherichia coli, Bacillus subtilis).

In some embodiments of the producer cell line of this invention, the heterologous nucleotide sequence can be integrated into the genome of the cells of the producer cell line.

In some embodiments of the producer cell line of this invention, the heterologous nucleotide sequence can be transiently present in the cells of the producer cell line.

In some embodiments, the produce cell line of this invention can comprise regulatory elements to control expression of the heterologous nucleotide sequence.

Nonlimiting examples of regulatory elements of this invention include regulatory elements for genetic control (e.g., a Cre/Lox system); for epigenetic control (e.g., acetylation, de-acetylation, methylation, de-methylation, ZFN, TALEN, CRISPR/Cas based activation or repression); for transcriptional control (e.g., a Dox on/off system, a Lac operon, an Aarabinose operon); for post-transcriptional control (e.g., degron, phospho-degron, a FKPB destabilization domain, a ribozyme, a light-inducible FOV based system, a small molecule based dimerizing domain); for translational control (e.g., IRES); for post-translational control (e.g., an intein system); and any combination thereof.

The present invention further comprises methods of producing virus particles (e.g., AAV1-10, Rhesus monkey AAV isolated, engineered AAV vectors) using the producer cell line of this invention.

### Definitions

The following terms are used in the description herein and the appended claims:

The singular forms “a,” “an” and “the” are intended to include the plural forms as well, unless the context clearly indicates otherwise.

Furthermore, the term “about,” as used herein when referring to a measurable value such as an amount of the length of a polynucleotide or polypeptide sequence, dose, time, temperature, and the like, is meant to encompass variations of ±20%, ±10%, ±5%, ±1%, ±0.5%, or even ±0.1% of the specified amount.

Also as used herein, “and/or” refers to and encompasses any and all possible combinations of one or more of the associated listed items, as well as the lack of combinations when interpreted in the alternative (“or”).

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 USPQ 461,463 (CCPA 1976) (emphasis in the original); see also MPEP § 2111.03. Thus, the term “consisting essentially of” when used in a claim of this invention is not intended to be interpreted to be equivalent to “comprising.”

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate further, if, for example, the specification indicates that a particular amino acid can be selected from A, G, I, L and/or V, this language also indicates that the amino acid can be selected from any subset of these amino acid(s) for example A, G, I or L; A, G, I or V; A or G; only L; etc. as if each such subcombination is expressly set forth herein. Moreover, such language also indicates that one or more of the specified amino acids can be disclaimed. For example, in particular embodiments the amino acid is not A, G or I; is not A; is not G or V; etc. as if each such possible disclaimer is expressly set forth herein.

As used herein, the terms “reduce,” “reduces,” “reduction” and similar terms mean a decrease of at least about 10%, 15%, 20%, 25%, 35%, 50%, 75%, 80%, 85%, 90%, 95%, 97% or more.

As used herein, the terms “enhance,” “enhances,” “enhancement” and similar terms indicate an increase of at least about 10%, 15%, 20%, 25%, 50%, 75%, 100%, 150%, 200%, 300%, 400%, 500% or more.

The term “parvovirus” as used herein encompasses the family Parvoviridae, including autonomously replicating parvoviruses and dependoviruses. The autonomous parvoviruses include members of the genera Protoparvovirus, Erythroparvovirus, Bocaparvirus, and Densovirus subfamily. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, muscovy duck parvovirus, B19 virus, and any other autonomous parvovirus now known or later discovered. Other autonomous parvoviruses are known to those skilled in the art. See, e.g., BERNARD N. FIELDS et al., VIROLOGY, volume 2, chapter 69 (4th ed., Lippincott-Raven Publishers; Cotmore et al. Archives of Virology DOI 10.1007/s00705-013-19144).

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAV type rh32.33, AAV type rh8, AAV type rh10, avian AAV, bovine AAV, canine AAV, equine AAV, ovine AAV, and any other AAV now known or later discovered. See, e.g., BERNARD N. FIELDS et al., VIROLOGY, volume 2, chapter 69 (4th ed., Lippincott-Raven Publishers). A number of AAV serotypes and clades have been identified (see, e.g., Gao et al., (2004) J. Virology 78:6381-6388; Moris et al., (2004) Virology 33-:375-383 and Table 1).

The genomic sequences of various serotypes of AAV and the autonomous parvoviruses, as well as the sequences of the native terminal repeats (TRs), Rep proteins, and capsid subunits are known in the art. Such sequences may be found in the literature or in public databases such as GenBank. See, e.g., GenBank Accession Numbers NC_002077, NC_001401, NC_001729, NC_001863, NC_001829, NC_001862, NC_000883, NC_001701, NC_001510, NC_006152, NC_006261, AF063497, U89790, AF043303, AF028705, AF028704, J02275, J01901, J02275, X01457, AF288061, AH009962, AY028226, AY028223, NC_001358, NC_001540, AF513851, AF513852, AY530579; the disclosures of which are incorporated by reference herein for teaching parvovirus and AAV nucleic acid and amino acid sequences. See also, e.g., Srivistava et al. (1983) J. Virology 45:555; Chiorini et al., (1998) J. Virology 71:6823; Chiorini et al., (1999)J. Virology 73:1309; Bantel-Schaal et al., (1999)J. Virology 73:939; Xiao et al., (1999)J. Virology 73:3994; Muramatsu et al., (1996) Virology 221:208; Shade et al., (1986) J. Virol. 58:921; Gao et al., (2002) Proc. Nat. Acad. Sci. USA 99:11854; Moris et al., (2004) Virology 33-:375-383; international patent publications WO 00/28061, WO 99/61601, WO 98/11244; and U.S. Pat. No. 6,156,303; the disclosures of which are incorporated by reference herein for teaching parvovirus and AAV nucleic acid and amino acid sequences. The capsid structures of autonomous parvoviruses and AAV are described in more detail in BERNARD N. FIELDS et al., VIROLOGY, volume 2, chapters 69 & 70 (4th ed., Lippincott-Raven Publishers). See also, description of the crystal structure of AAV2 (Xie et al., (2002) Proc. Nat. Acad. Sci. 99:10405-10), AAV9 (DiMattia et al., (2012) J. Virol. 86:6947-6958), AAV8 (Nam et al., (2007) J. Virol. 81:12260-12271), AAV6 (Ng et al., (2010) J. Virol. 84:12945-12957), AAV5 (Govindasamy et al., (2013) J. Virol. 87, 11187-11199), AAV4 (Govindasamy et al., (2006) J. Virol. 80:11556-11570), AAV3B (Lerch et al., (2010) Virology 403: 26-36), BPV (Kailasan et al., (2015) J. Virol. 89:2603-2614) and CPV (Xie et al., (1996) J. Mol. Biol. 6:497-520 and Tsao et al., (1991) Science 251: 1456-64).

The term “tropism” as used herein refers to preferential entry of the virus into certain cells or tissues, optionally followed by expression (e.g., transcription and, optionally, translation) of a sequence(s) carried by the viral genome in the cell, e.g., for a recombinant virus, expression of a heterologous nucleic acid(s) of interest.

Those skilled in the art will appreciate that transcription of a heterologous nucleic acid sequence from the viral genome may not be initiated in the absence of transacting factors, e.g., for an inducible promoter or otherwise regulated nucleic acid sequence. In the case of a rAAV genome, gene expression from the viral genome may be from a stably integrated provirus, from a non-integrated episome, as well as any other form in which the virus may take within the cell.

As used here, “systemic tropism” and “systemic transduction” (and equivalent terms) indicate that the virus capsid or virus vector of the invention exhibits tropism for or transduces, respectively, tissues throughout the body (e.g., brain, lung, skeletal muscle, heart, liver, kidney and/or pancreas). In embodiments of the invention, systemic transduction of muscle tissues (e.g., skeletal muscle, diaphragm and cardiac muscle) is observed. In other embodiments, systemic transduction of skeletal muscle tissues achieved. For example, in particular embodiments, essentially all skeletal muscles throughout the body are transduced (although the efficiency of transduction may vary by muscle type). In particular embodiments, systemic transduction of limb muscles, cardiac muscle and diaphragm muscle is achieved. Optionally, the virus capsid or virus vector is administered via a systemic route (e.g., systemic route such as intravenously, intra-articularly or intra-lymphatically). Alternatively, in other embodiments, the capsid or virus vector is delivered locally (e.g., to the footpad, intramuscularly, intradermally, subcutaneously, topically).

Unless indicated otherwise, “efficient transduction” or “efficient tropism,” or similar terms, can be determined by reference to a suitable control (e.g., at least about 50%, 60%, 70%, 80%, 85%, 90%, 95% or more of the transduction or tropism, respectively, of the control). In particular embodiments, the virus vector efficiently transduces or has efficient tropism for skeletal muscle, cardiac muscle, diaphragm muscle, pancreas (including (3-islet cells), spleen, the gastrointestinal tract (e.g., epithelium and/or smooth muscle), cells of the central nervous system, lung, joint cells, and/or kidney. Suitable controls will depend on a variety of factors including the desired tropism profile. For example, AAV8 and AAV9 are highly efficient in transducing skeletal muscle, cardiac muscle and diaphragm muscle, but have the disadvantage of also transducing liver with high efficiency.

Similarly, it can be determined if a virus “does not efficiently transduce” or “does not have efficient tropism” for a target tissue, or similar terms, by reference to a suitable control. In particular embodiments, the virus vector does not efficiently transduce (i.e., has does not have efficient tropism) for liver, kidney, gonads and/or germ cells. In particular embodiments, undesirable transduction of tissue(s) (e.g., liver) is 20% or less, 10% or less, 5% or less, 1% or less, 0.1% or less of the level of transduction of the desired target tissue(s) (e.g., skeletal muscle, diaphragm muscle, cardiac muscle and/or cells of the central nervous system).

As used herein, the term “polypeptide” encompasses both peptides and proteins, unless indicated otherwise.

A “polynucleotide” is a sequence of nucleotide bases, and may be RNA, DNA or DNA-RNA hybrid sequences (including both naturally occurring and non-naturally occurring nucleotide), but in representative embodiments are either single or double stranded DNA sequences.

As used herein, an “isolated” polynucleotide (e.g., an “isolated DNA” or an ‘isolated RNA”) means a polynucleotide at least partially separated from at least some of the other components of the naturally occurring organism or virus, for example, the cell or viral structural components or other polypeptides or nucleic acids commonly found associated with the polynucleotide. In representative embodiments an “isolated” nucleotide is enriched by at least about 10-fold, 100-fold, 1000-fold, 10,000-fold or more as compared with the starting material.

Likewise, an “isolated” polypeptide means a polypeptide that is at least partially separated from at least some of the other components of the naturally occurring organism or virus, for example, the cell or viral structural components or other polypeptides or nucleic acids commonly found associated with the polypeptide. In representative embodiments an “isolated” polypeptide is enriched by at least about 10-fold, 100-fold, 1000-fold, 10,000-fold or more as compared with the starting material.

As used herein, by “isolate” or “purify” (or grammatical equivalents) a virus vector, it is meant that the virus vector is at least partially separated from at least some of the other components in the starting material. In representative embodiments an “isolated” or “purified” virus vector is enriched by at least about 10-fold, 100-fold, 1000-fold, 10,000-fold or more as compared with the starting material.

A “therapeutic polypeptide” is a polypeptide that can alleviate, reduce, prevent, delay and/or stabilize symptoms that result from an absence or defect in a protein in a cell or subject and/or is a polypeptide that otherwise confers a benefit to a subject, e.g., anti-cancer effects or improvement in transplant survivability.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

The terms “heterologous nucleotide sequence” and “heterologous nucleic acid” are used interchangeably herein and refer to a sequence that is not naturally occurring in the virus. Generally, the heterologous nucleic acid comprises an open reading frame that encodes a polypeptide or nontranslated RNA of interest (e.g., for delivery to a cell or subject).

As used herein, the terms “virus vector,” “vector” or “gene delivery vector” refer to a virus (e.g., AAV) particle that functions as a nucleic acid delivery vehicle, and which comprises the vector genome (e.g., viral DNA [vDNA]) packaged within a virion. Alternatively, in some contexts, the term “vector” may be used to refer to the vector genome/vDNA alone.

A “rAAV vector genome” or “rAAV genome” is an AAV genome (i.e., vDNA) that comprises one or more heterologous nucleic acid sequences. rAAV vectors generally require only the terminal repeat(s) (TR(s)) in cis to generate virus. All other viral sequences are dispensable and may be supplied in trans (Muzyczka, (1992) Curr. Topics Microbiol. Immunol. 158:97). Typically, the rAAV vector genome will only retain the one or more TR sequence so as to maximize the size of the transgene that can be efficiently packaged by the vector. The structural and non-structural protein coding sequences may be provided in trans (e.g., from a vector, such as a plasmid, or by stably integrating the sequences into a packaging cell). In embodiments of the invention the rAAV vector genome comprises at least one TR sequence (e.g., AAV TR sequence), optionally two TRs (e.g., two AAV TRs), which typically will be at the 5′ and 3′ ends of the vector genome and flank the heterologous nucleic acid, but need not be contiguous thereto. The TRs can be the same or different from each other.

The term “terminal repeat” or “TR” includes any viral terminal repeat or synthetic sequence that forms a hairpin structure and functions as an inverted terminal repeat (i.e., mediates the desired functions such as replication, virus packaging, integration and/or provirus rescue, and the like). The TR can be an AAV TR or a non-AAV TR. For example, a non-AAV TR sequence such as those of other parvoviruses (e.g., canine parvovirus (CPV), mouse parvovirus (MVM), human parvovirus B-19) or any other suitable virus sequence (e.g., the SV40 hairpin that serves as the origin of SV40 replication) can be used as a TR, which can further be modified by truncation, substitution, deletion, insertion and/or addition. Further, the TR can be partially or completely synthetic, such as the “double-D sequence” as described in U.S. Pat. No. 5,478,745 to Samulski et al.

An “AAV terminal repeat” or “AAV TR” may be from any AAV, including but not limited to serotypes 1, 2, 3, 4, 5, 6, 7, 8, 9, 10, 11, 12, 13 or any other AAV now known or later discovered. An AAV terminal repeat need not have the native terminal repeat sequence (e.g., a native AAV TR sequence may be altered by insertion, deletion, truncation and/or missense mutations), as long as the terminal repeat mediates the desired functions, e.g., replication, virus packaging, integration, and/or provirus rescue, and the like.

The virus vectors of the invention can further be “targeted” virus vectors (e.g., having a directed tropism) and/or a “hybrid” parvovirus (i.e., in which the viral TRs and viral capsid are from different parvoviruses) as described in international patent publication WO 00/28004 and Chao et al., (2000) Molecular Therapy 2:619.

The virus vectors of the invention can further be duplexed parvovirus particles as described in international patent publication WO 01/92551 (the disclosure of which is incorporated herein by reference in its entirety). Thus, in some embodiments, double stranded (duplex) genomes can be packaged into the virus capsids of the invention.

Further, the viral capsid or genomic elements can contain other modifications, including insertions, deletions and/or substitutions.

As used herein, the term “amino acid” encompasses any naturally occurring amino acid, modified forms thereof, and synthetic amino acids. Naturally occurring, levorotatory (L−) amino acids are shown in Table 2.

Alternatively, the amino acid can be a modified amino acid residue (nonlimiting examples are shown in Table 4) and/or can be an amino acid that is modified by post-translation modification (e.g., acetylation, amidation, formylation, hydroxylation, methylation, phosphorylation or sulfatation).

Further, the non-naturally occurring amino acid can be an “unnatural” amino acid as described by Wang et al., Annu Rev Biophys Biomol Struct. 35:225-49 (2006)). These unnatural amino acids can advantageously be used to chemically link molecules of interest to the AAV capsid protein.

Methods of determining sequence similarity or identity between two or more amino acid sequences are known in the art. Sequence similarity or identity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J. Mol. Biol. 48,443 (1970), by the search for similarity method of Pearson & Lipman, Proc. Natl. Acad. Sci. USA 85,2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12, 387-395 (1984), or by inspection.

Another suitable algorithm is the BLAST algorithm, described in Altschul et al., J. Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460-480 (1996); http://blast.wustl/edu/blast/README.html. WU-BLAST-2 uses several search parameters, which are optionally set to the default values. The parameters are dynamic values and are established by the program itself depending upon the composition of the particular sequence and composition of the particular database against which the sequence of interest is being searched; however, the values may be adjusted to increase sensitivity.

Further, an additional useful algorithm is gapped BLAST as reported by Altschul et al., (1997) Nucleic Acids Res. 25, 3389-3402.

Those skilled in the art will appreciate that for some AAV capsid proteins the corresponding modification can be an insertion and/or a substitution, depending on whether the corresponding amino acid positions are partially or completely present in the virus or, alternatively, are completely absent. As one nonlimiting example, when modifying AAV other than AAV2, the specific amino acid position(s) may be different than the position in AAV2, thereby identifying the corresponding amino acid position in a different AAV serotype (see, e.g., Table 3). The corresponding amino acid position(s) will be readily apparent to those skilled in the art using well-known techniques, including for example, alignment of sequences.

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to an immunogen by infection or by vaccination. Active immunity can be contrasted with passive immunity, which is acquired through the “transfer of preformed substances (antibody, transfer factor, thymic graft, interleukin-2) from an actively immunized host to a non-immune host.” Id.

A “protective” immune response or “protective” immunity as used herein indicates that the immune response confers some benefit to the subject in that it prevents or reduces the incidence of disease. Alternatively, a protective immune response or protective immunity may be useful in the treatment and/or prevention of disease, in particular cancer or tumors (e.g., by preventing cancer or tumor formation, by causing regression of a cancer or tumor and/or by preventing metastasis and/or by preventing growth of metastatic nodules). The protective effects may be complete or partial, as long as the benefits of the treatment outweigh any disadvantages thereof.

It is known in the art that immune responses may be enhanced by immunomodulatory cytokines (e.g., α-interferon, β-interferon, γ-interferon, ω-interferon, τ-interferon, interleukin-1α, interleukin-1β, interleukin-2, interleukin-3, interleukin-4, interleukin 5, interleukin-6, interleukin-7, interleukin-8, interleukin-9, interleukin-10, interleukin-11, interleukin 12, interleukin-1β, interleukin-14, interleukin-18, B cell Growth factor, CD40 Ligand, tumor necrosis factor-α, tumor necrosis factor-β, monocyte chemoattractant protein-1, granulocyte-macrophage colony stimulating factor, and lymphotoxin). Accordingly, immunomodulatory cytokines (preferably, CTL inductive cytokines) may be administered to a subject in conjunction with the virus vector.

Cytokines may be administered by any method known in the art. Exogenous cytokines may be administered to the subject, or alternatively, a nucleic acid encoding a cytokine may be delivered to the subject using a suitable vector, and the cytokine produced in vivo.

The term “avian” as used herein includes, but is not limited to, chickens, ducks, geese, quail, turkeys, pheasant, parrots, parakeets, and the like. The term “mammal” as used herein includes, but is not limited to, humans, non-human primates, bovines, ovines, caprines, equines, felines, canines, lagomorphs, etc. Human subjects include neonates, infants, juveniles, adults and geriatric subjects.

In representative embodiments, the subject is “in need of” the methods of the invention.

By “pharmaceutically acceptable” it is meant a material that is not toxic or otherwise undesirable, i.e., the material may be administered to a subject without causing any undesirable biological effects.

Methods of Producing Virus Vectors.

In one embodiment, the present invention provides a method of producing a virus particle, comprising providing to a producer cell of this invention comprising a nucleotide sequence encoding an engineered AAP of this invention: (a) a nucleic acid template comprising at least one TR sequence (e.g., AAV TR sequence), and (b) AAV sequences sufficient for replication of the nucleic acid template and encapsidation into AAV capsids (e.g., AAV rep sequences and AAV cap sequences encoding the AAV capsids of the invention and/or the engineered AAP of this invention). Optionally, the nucleic acid template further comprises at least one heterologous nucleic acid sequence. In particular embodiments, the nucleic acid template comprises two AAV ITR sequences, which are located 5′ and 3′ to the heterologous nucleic acid sequence (if present), although they need not be directly contiguous thereto. In some embodiments, the producer cell can comprise helper nucleic acid (e.g., a plasmid) comprising adenoviral genes.

The nucleic acid template and AAV rep and cap sequences are provided under conditions such that virus particle comprising the nucleic acid template packaged within the AAV capsid is produced in the cell. The method can further comprise the step of collecting the virus particle from the cell. The virus particles can be collected from the medium and/or by lysing the cells.

The cell can be a cell that is permissive for AAV viral replication. Any suitable cell known in the art may be employed. In particular embodiments, the cell is a mammalian cell. As another option, the cell can be a trans-complementing packaging cell line that provides functions deleted from a replication-defective helper virus, e.g., 293 cells or other Ela and/or E1b trans-complementing cells.

The AAV replication and capsid sequences and the engineered AAP sequences may be provided by any method known in the art. In some embodiments, current protocols express the AAV rep/cap genes on a single plasmid. The AAV replication and packaging sequences and AAP sequences need not be provided together, although it may be convenient to do so. The AAV rep and/or cap sequences and/or AAP sequences may be provided by any viral or non-viral vector. For example, the rep/cap sequences may be provided by a hybrid adenovirus or herpesvirus vector (e.g., inserted into the E1a or E3 regions of a deleted adenovirus vector). EBV vectors may also be employed to express the AAV cap and rep genes and/or AAP sequence. One advantage of this method is that EBV vectors are episomal, yet will maintain a high copy number throughout successive cell divisions (i.e., are stably integrated into the cell as extra-chromosomal elements, designated as an “EBV based nuclear episome,” see Margolski, (1992) Curr. Top. Microbiol. Immun. 158:67).

As a further alternative, the rep/cap sequences and/or AAP sequences may be stably incorporated into a cell.

Typically the AAV rep/cap sequences will not be flanked by the TRs, to prevent rescue and/or packaging of these sequences.

The nucleic acid template can be provided to the cell using any method known in the art. For example, the template can be supplied by a non-viral (e.g., plasmid) or viral vector. In particular embodiments, the nucleic acid template is supplied by a herpesvirus or adenovirus vector (e.g., inserted into the E1a or E3 regions of a deleted adenovirus). As another illustration, Palombo et al., (1998) J. Virology 72:5025, describes a baculovirus vector carrying a reporter gene flanked by the AAV TRs. EBV vectors may also be employed to deliver the template, as described above with respect to the rep/cap genes.

In another representative embodiment, the nucleic acid template is provided by a replicating rAAV virus. In still other embodiments, an AAV provirus comprising the nucleic acid template is stably integrated into the chromosome of the cell.

To enhance virus titers, helper virus functions (e.g., adenovirus or herpesvirus) that promote a productive AAV infection can be provided to the cell. Helper virus sequences necessary for AAV replication are known in the art. Typically, these sequences will be provided by a helper adenovirus or herpesvirus vector. Alternatively, the adenovirus or herpesvirus sequences can be provided by another non-viral or viral vector, e.g., as a non-infectious adenovirus miniplasmid that carries all of the helper genes that promote efficient AAV production as described by Ferrari et al., (1997) Nature Med. 3:1295, and U.S. Pat. Nos. 6,040,183 and 6,093,570.

Further, the helper virus functions may be provided by a producer cell with the helper sequences embedded in the chromosome or maintained as a stable extrachromosomal element. Generally, the helper virus sequences cannot be packaged into AAV particles, e.g., are not flanked by TRs.

Those skilled in the art will appreciate that it may be advantageous to provide the AAV replication and capsid sequences and the helper virus sequences (e.g., adenovirus sequences) on a single helper construct. This helper construct may be a non-viral or viral construct. As one nonlimiting illustration, the helper construct can be a hybrid adenovirus or hybrid herpesvirus comprising the AAV rep/cap genes.

In one particular embodiment, the AAV rep/cap sequences and the adenovirus helper sequences are supplied by a single adenovirus helper vector. This vector can further comprise the nucleic acid template. The AAV rep/cap sequences and/or the rAAV template can be inserted into a deleted region (e.g., the E1a or E3 regions) of the adenovirus.

In a further embodiment, the AAV rep/cap sequences and the adenovirus helper sequences are supplied by a single adenovirus helper vector. According to this embodiment, the rAAV template can be provided as a plasmid template.

In another illustrative embodiment, the AAV rep/cap sequences and adenovirus helper sequences are provided by a single adenovirus helper vector, and the rAAV template is integrated into the cell as a provirus. Alternatively, the rAAV template is provided by an EBV vector that is maintained within the cell as an extrachromosomal element (e.g., as an EBV based nuclear episome).

In a further exemplary embodiment, the AAV rep/cap sequences and adenovirus helper sequences are provided by a single adenovirus helper. The rAAV template can be provided as a separate replicating viral vector. For example, the rAAV template can be provided by a rAAV particle or a second recombinant adenovirus particle.

According to the foregoing methods, the hybrid adenovirus vector typically comprises the adenovirus 5′ and 3′ cis sequences sufficient for adenovirus replication and packaging (i.e., the adenovirus terminal repeats and PAC sequence). The AAV rep/cap sequences and, if present, the rAAV template are embedded in the adenovirus backbone and are flanked by the 5′ and 3′ cis sequences, so that these sequences may be packaged into adenovirus capsids. As described above, the adenovirus helper sequences and the AAV rep/cap sequences are generally not flanked by TRs so that these sequences are not packaged into the AAV virions.

Zhang et al., ((2001) Gene Ther. 18:704-12) describe a chimeric helper comprising both adenovirus and the AAV rep and cap genes.

Herpesvirus may also be used as a helper virus in AAV packaging methods. Hybrid herpesviruses encoding the AAV Rep protein(s) may advantageously facilitate scalable AAV vector production schemes. A hybrid herpes simplex virus type I (HSV-1) vector expressing the AAV-2 rep and cap genes has been described (Conway et al., (1999) Gene Therapy 6:986 and WO 00/17377.

As a further alternative, the virus vectors of the invention can be produced in insect cells using baculovirus vectors to deliver the rep/cap genes and rAAV template as described, for example, by Urabe et al., (2002) Human Gene Therapy 13:1935-43.

AAV vector stocks free of contaminating helper virus may be obtained by any method known in the art. For example, AAV and helper virus may be readily differentiated based on size. AAV may also be separated away from helper virus based on affinity for a heparin substrate (Zolotukhin et al. (1999) Gene Therapy 6:973). Deleted replication-defective helper viruses can be used so that any contaminating helper virus is not replication competent. As a further alternative, an adenovirus helper lacking late gene expression may be employed, as only adenovirus early gene expression is required to mediate packaging of AAV virus.

Adenovirus mutants defective for late gene expression are known in the art (e.g., ts100K and ts149 adenovirus mutants).

In some embodiments, expression of AAP coding sequences in trans using any of the methods described herein and/or as otherwise known in the art can be used to rescue assembly of an otherwise defective AAV capsid protein carrying mutations or deletions or insertions in the Cap gene region that overlaps with the AAP sequence. In some embodiments, mutations can be introduced in the AAP coding region of the Cap gene that result in blocking capsid assembly and expressing the engineered AAP sequence in trans can rescue this defect in assembly.

The present subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### Examples

The following EXAMPLES provide illustrative embodiments. Certain aspects of the following EXAMPLES are disclosed in terms of techniques and procedures found or contemplated by the present inventors to work well in the practice of the embodiments. In light of the present disclosure and the general level of skill in the art, those of skill will appreciate that the following EXAMPLES are intended to be exemplary only and that numerous changes, modifications, and alterations can be employed without departing from the scope of the presently claimed subject matter.

### Example 1. Mapping and Engineering Functional Domains of the Assembly Activating Protein (AAP) of Adeno-Associated Viruses

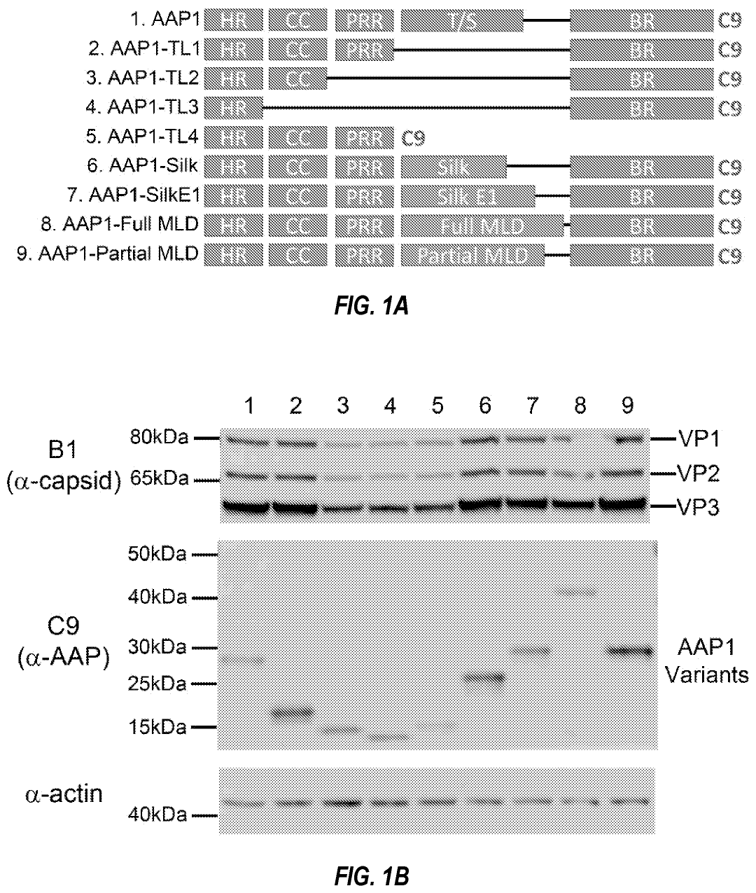

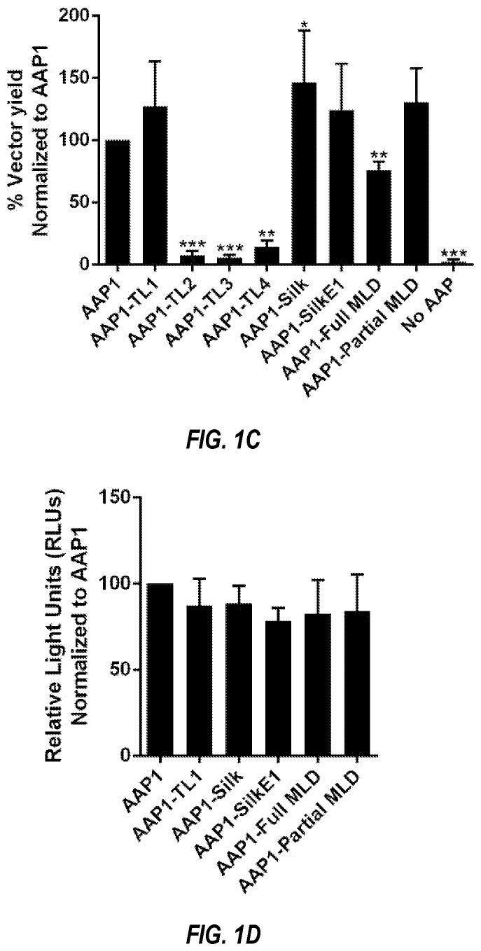

The T/S region of AAP is a flexible linker that can be replaced by different structures. We generated a series of deletion mutants based on AAP domains and tested for the function of AAP using four parameters: steady state AAP levels, VP levels, vector yield and transduction efficiency. All AAP constructs have a C9-tag derived from bovine rhodopsin at the C-terminus for immunodetection (FIG. 1A). In brief, experiments were performed by transfecting pXX680, pTR-CBA-Luc, pXR1ΔAAP and a pCDNA3.1-AAP construct into HEK293 cells. All deletion and the T/S replacement constructs produced AAP at different levels. While truncated linker 2 (TL2), truncated linker 3 (TL3) and the T/S replaced with Bombyx mori silk heavy chain repeats (Silk and SilkE1) or the entire mucin-like domain from murine alpha dystroglycan mucin like domain (Full MLD) showed similar steady levels of AAP, truncated linker 1 (TL1) and T/S replaced by partial mucin-like domain (Partial MLD) produce significantly more AAP. Truncated linker 4 (TL4) shows a reduced level of AAP expression (FIG. 1B, Table 6). Multiple reports have shown that one of the observable function of AAP is to help stabilizing/expression of VP; therefore, we next determined the steady state VP level in the transfected cells. The level of VP3 correlates proportionally with AAP protein level, except for mutants TL2 and TL3 which have a significant reduction in VP3 level compared to WT AAP, although they produced a similar amount of AAP (FIG. 1B). Despite the production of both AAP and VP proteins in all condition tested, deletion of the HR, CC or PRR regions rendered AAP incapable of supporting AAV capsid assembly (FIG. 1C, Table 6). However, deletion of the entire T/S or replacing the region with other flexible protein domains does not affect any tested function of AAP (including capsid assembly) strongly suggesting the T/S is non-essential for the assembly function of AAP (FIG. 1C, Table 6). The transduction efficiency of the recombinant AAV vectors produced using different AAP constructs were similar to control. Based on these findings, we concluded that the T/S region can potentially be engineered with other heterologous sequences to generate engineered AAPs (eAAP)s with new functions.

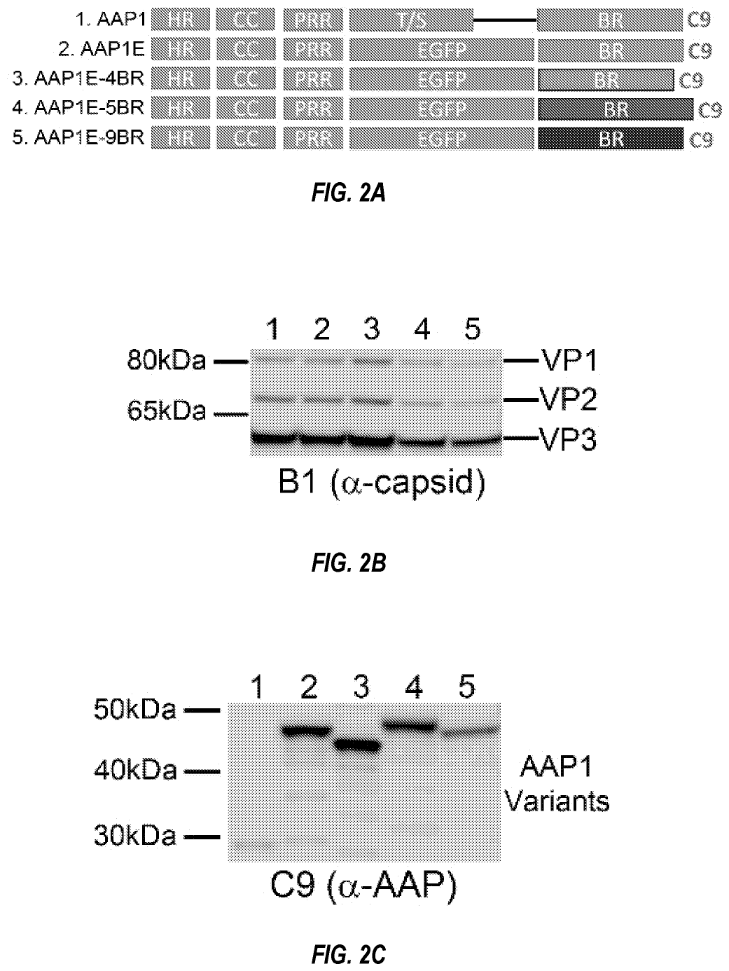

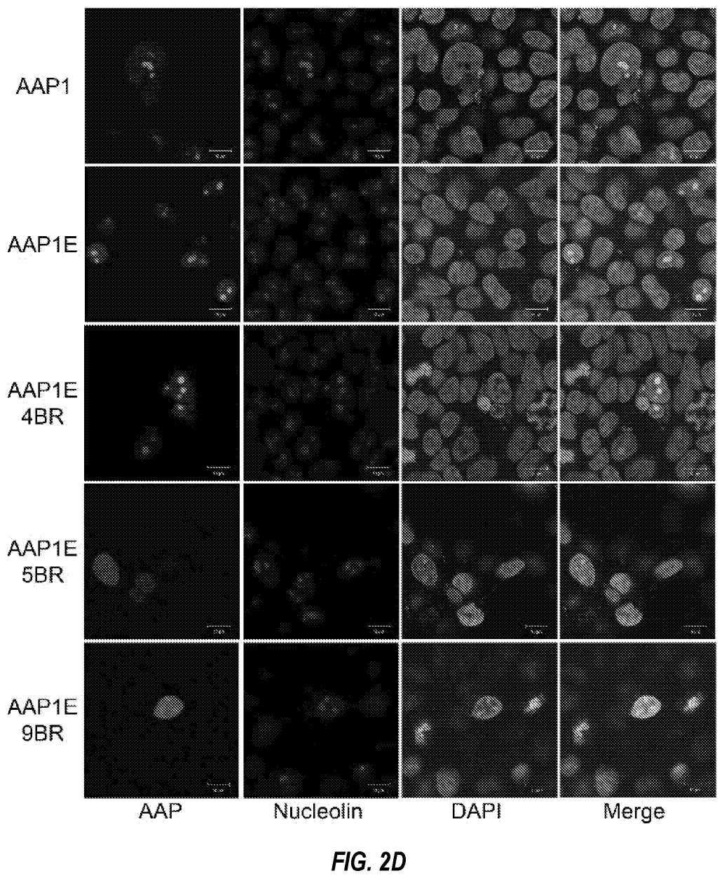

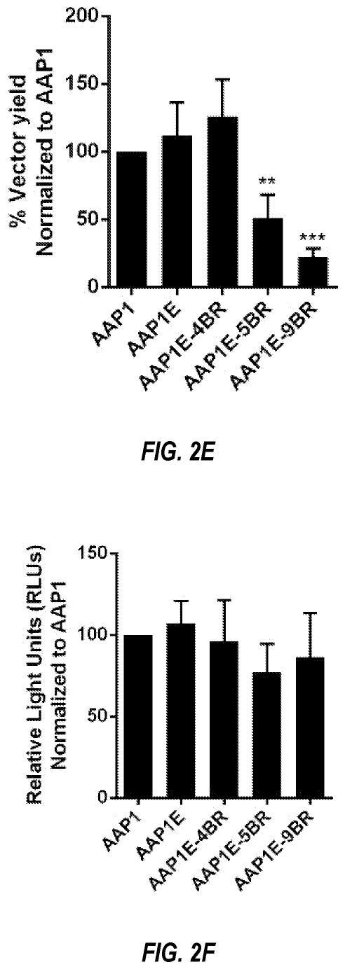

The basic region (BR) of AAP is not serotype specific. We first created a traceable AAP1 protein by replacing the T/S with EGFP to create AAP1-EGFP (AAP1E) for fluorescent tracking (FIG. 2A). This newly engineered AAP1E shows the same level of VP expression and capsid assembly, but a higher level of AAP expression compared to AAP1 (FIGS. 2B, 2C, and 2E, Table 7). Furthermore, the AAP1E mutant localizes within the nucleolus similar to the wild type AAP1, suggesting AAP1E can be useful for further mechanistic studies (FIG. 2D). Using the AAP1E construct, we swapped the BR region of AAP1E with the BR from AAP-4, -5 and -9 (FIG. 2A). The VP and AAP expression profiles of the BR substitution mutants are generally similar to AAP1E. However, 5BR and 9BR show a slight decrease in VP expression and 9BR shows a slight decrease in AAP expression (FIGS. 2B and 2C, Table 7). Notably, BR1 and BR4 strongly localized to the nucleolus, while BR5 and BR9 only shows partial nucleolus localization as reported earlier (FIG. 2D, Table 7). Intracellular localization appears to correlate with vector titers, for instance, BR5 and BR9 only restore about 50% of assembly (FIG. 2F, Table 7). In contrast, BR4 is able to restore AAP1 function to 120% compared to WT AAP1. Transduction efficiency of the rAAVs generated using different AAPs was not affected (FIG. 2F). As it is known that AAP4 cannot rescue AAV1 capsid assembly, our data suggests that serotype specific trans-complementation is not due to the BR, but rather other regions of AAP.

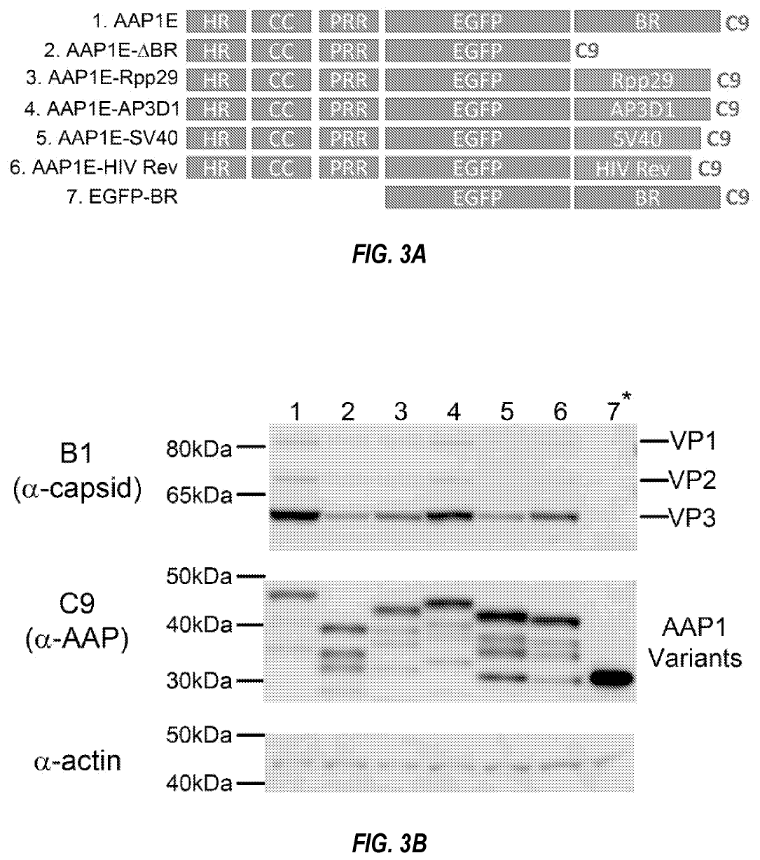

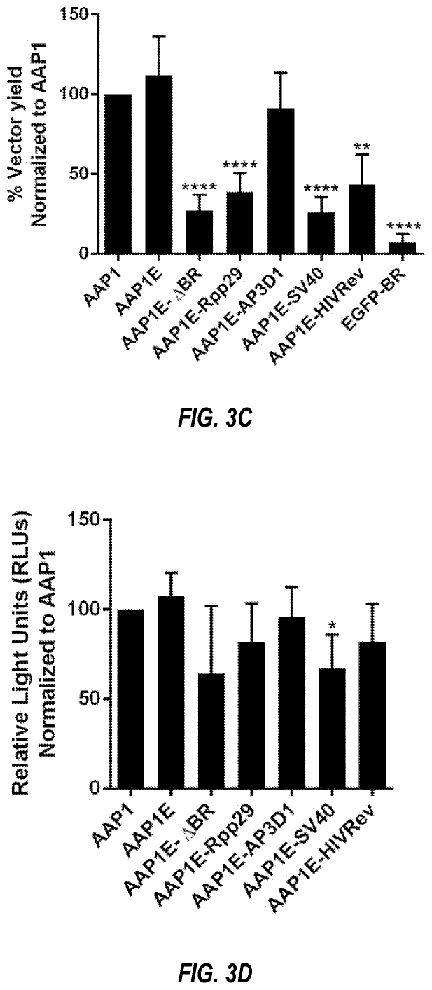

The basic region of AAP1 can be replaced by other nucleolar localization signals (NoLS) without disrupting function. Based on the above results, we hypothesized that the BR region can be replaced completely by other NoLS without affecting AAP function. Therefore, we engineered AAP1E by replacing the BR with other known nuclear or nucleolar localization signals (NLS/NoLS). The amino acid sequences and annotation of each NLS/NoLS are described in Table 5. In brief, we picked 4 different NLS/NoLS peptides from the ribonuclease P subunit NoLS (Rpp29), adaptor protein in AP-3 complex (AP3D1), viral derived SV40 NLS (SV40) and HIV Rev NoLS domain (HIV Rev) (FIG. 3A).

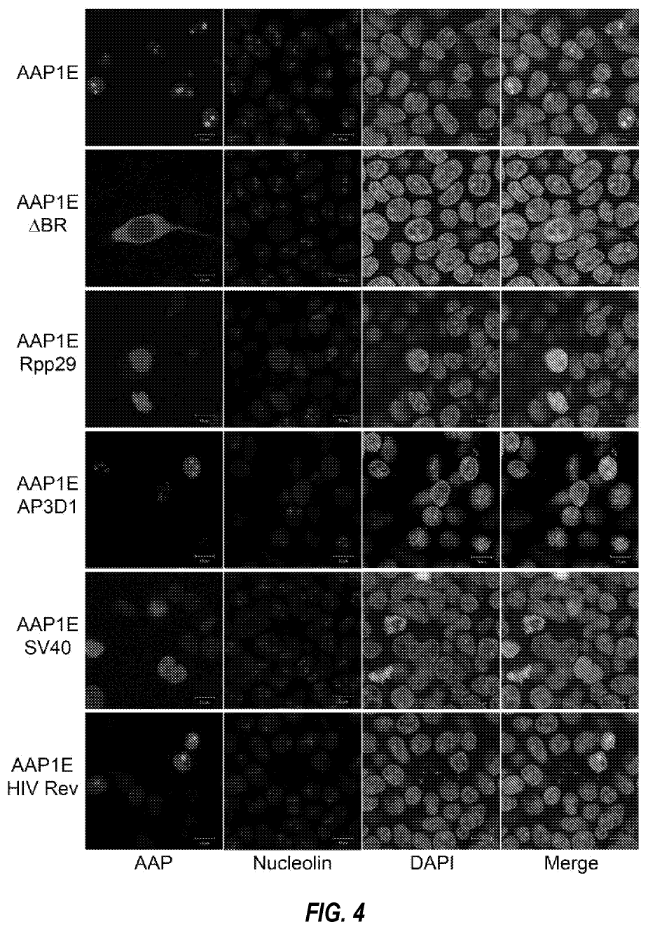

Although AAP expression level are comparable to AAP1E from all the BR replacement mutants, VP expression was moderately reduced for some (FIG. 3B, Table 7). This decrease correlates with assembly based on vector yield, where the various BR mutants show a spectrum of activity. SV40 is unable to restore AAP assembly function, while Rpp29, HIV-Rev and AP3D1 restore AAP assembly function at 40%, 75% and 80% respectively (FIG. 3C, Table 7). All rAAV generated with these constructs show similar transduction efficiency (FIG. 3D, Table 7). Since the NoLS/NLS mutants showed a spectrum of AAP function, we performed confocal microscopy to investigate trafficking efficiency compared to AAP. AAP1E shows clear co-localization with the nucleolar marker CD23 (FIG. 4). AAP1E-ΔBR shows cytoplasmic localization as expected, and is not able to restore function (FIG. 3C and FIG. 4). AAP1E-SV40 shows nuclear localization, which only partially rescues AAV assembly. AAP1E-Rpp29 shows moderate (40%) rescue of AAV assembly, and has partial nucleolar localization (FIG. 3C and FIG. 4). HIV-Rev and AP3D1 show nucleolar localization and AP3D1 restores assembly function completely (FIG. 3C and FIG. 4). Therefore, the ability of AAP to drive AAV1 assembly correlates with its nuclear/nucleolar localization and the BR region can be replaced by other NoLS signals without affecting AAP function.

Functionally mapping the structural domains at the N-terminus of AAP1E.

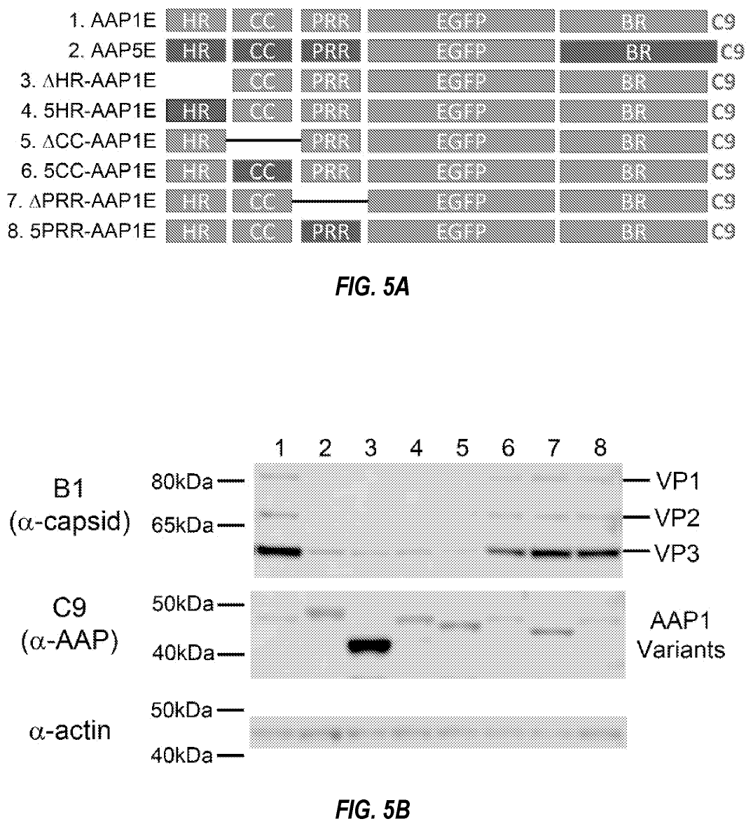

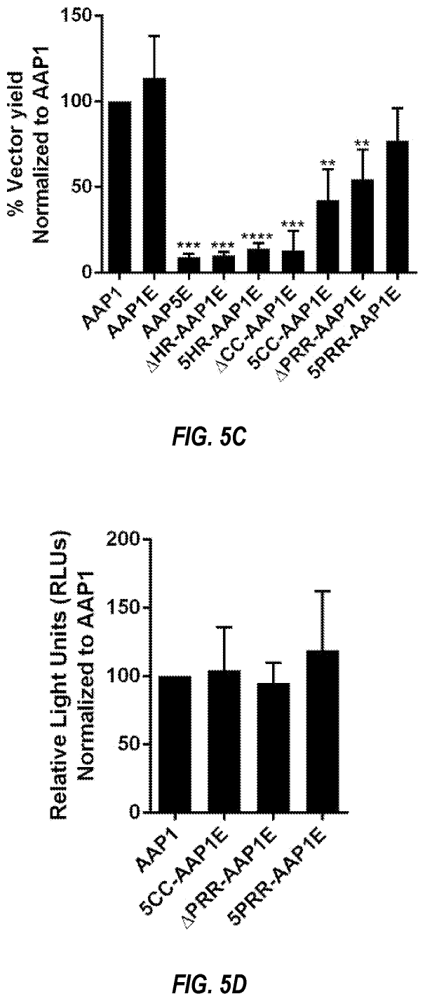

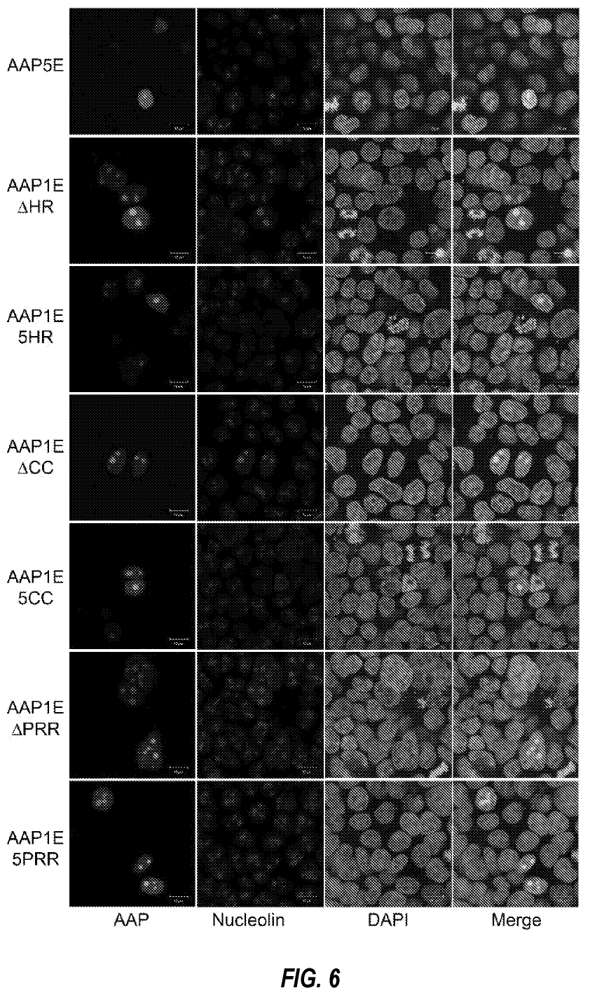

Currently, there is no structural information on AAP. Based on a modular homology modelling, the N-terminal HR and CC domains are the only regions that have defined structure and were modeled separately. The HR region is modeled as an alpha-helix and the CC region is modeled as either a loop or a beta strand. Deletion or point mutations at these regions completely abolish the ability of AAP to activate capsid assembly suggesting that these secondary structures play an important role in AAP functions. Furthermore, previous reports have shown that AAP5 is unable to support capsid assembly of AAV1, despite the sequence similarity between PRR, T/S and BR regions. We investigated the function of each sub-domain by deletion analysis and by replacement with the corresponding domain of AAP5. (FIG. 5A). While all the constructs were able to produce AAP, the functional efficiencies were varied. The expression level of the AAP1EΔHR construct is higher than the AAP1E; however, the level of VP is significantly reduced (FIG. 5B, Table 6). Additionally, capsid formation is completely abrogated to background levels (FIG. 5C, Table 6). Replacing the AAP1 HR domain with the AAP5 HR domain shows a similar effect as the deletion, although the AAP expression level is only slightly increased compared to AAP1E (FIGS. 5B and 5C, Table 6). Deletion of the CC region resulted in a similar phenotype, where the expression level of AAP was normal, but VP expression and capsid formation were reduced to background level (FIGS. 5B and 5C, Table 6). Replacing 1CC with 5CC restored the level of VP expression and 70% of capsid assembly, suggesting a connection between CC region and capsid protein stability that is not serotype specific (FIGS. 5B and 5C, Table 6). Deletion of the PRR region shows a moderate effect on capsid expression and assembly. Replacing the 1PRR region with 5PRR only slight affect AAP function and allow 75% of capsid assembly (FIGS. 5B and 5C, Table 6). All rAAV generated with AAP show similar transduction efficiency (FIG. 5D). Confocal microcopy studies further confirmed that the N-terminal modules localized at the nucleolus similar to the AAP1E control (FIG. 6). These results corroborate the notion that the HR and CC domains are essential for VP interactions and maintaining stability.

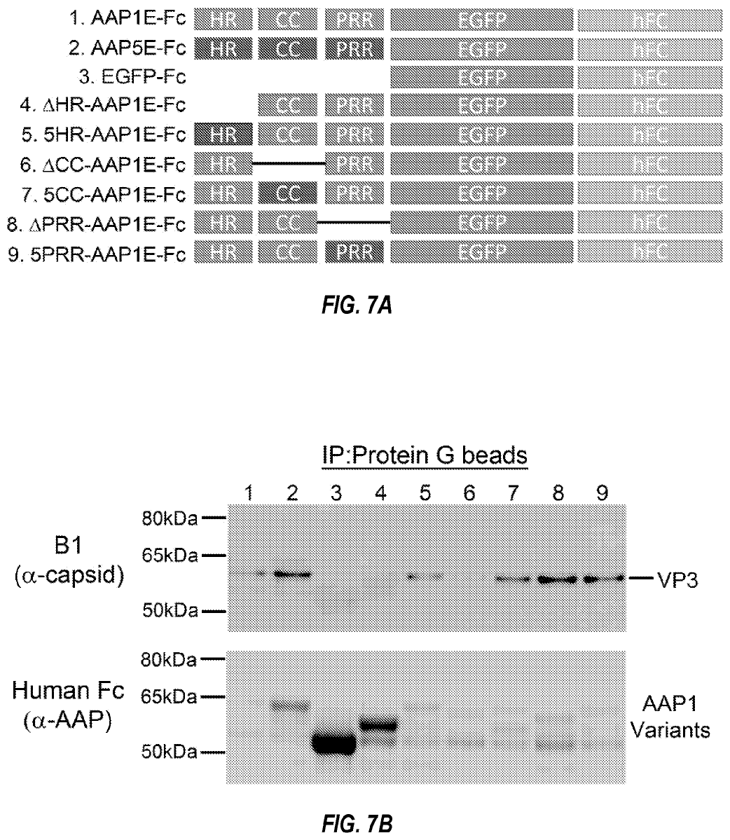

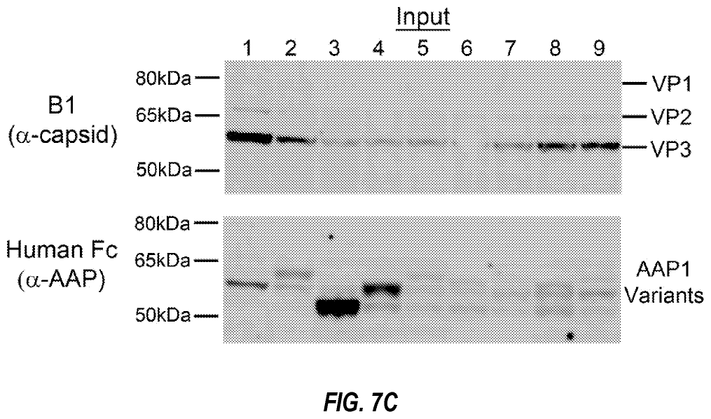

The HR and CC domains are the major structural determinants driving AAP-capsid interactions. Based on deletion and AAP1/5 chimera analysis, we established that capsid assembly associates tightly with VP expression. We utilized the same deletion and replacement constructs as above and replaced the BR region with a human Fc domain to facilitate VP complexation and immunoprecipitation studies (FIG. 7A). The expression level of both VP and AAP varies between constructs. For instance, EGFP, ΔHR, 5HR, ΔCC and 5CC show a reduced level of AAP expression, while AAP5E and ΔHR show an increase in AAP expression (FIG. 7C, Table 6). To verify our system, we first showed that AAP1E can pull down the AAV1 VP. Surprisingly, although AAP5E was unable to support AAV1 capsid assembly (FIG. 6C), this protein was able to pull down AAV1 VP with only a slight reduction compared to AAP1E (FIG. 7B, Table 6). Deletion of the HR or CC regions abolish the ability of AAP to pull down VP. Replacing the HR and CC regions with AAP5 restores the ability of AAP to pull down AAV1 VP (FIG. 7B, Table 6). Deletion of PRR or replacing 1PRR with 5PRR have no significant effect on AAV1 capsid interaction (FIG. 7B, Table 6). It is worth noting that the lack of immunoprecipitation in some of these construct cannot be explained by expression level differences. For instance, 5HR and 5CC both have low levels of AAP and VP expression, but are able to pull down VP in our assay (FIGS. 7B and 7C, Table 6). On the other hand, ΔHR and the EGFP control are unable to pull down any VP protein despite having the highest levels of AAP expression (FIGS. 7B and 7C, Table 6). However, this interaction is either transient or weak; which is further supported by our observation that majority of the capsid protein are still found in the flow through fraction of the pull down. These results demonstrate that the interactions between AAP and VP are driven by the N-terminal modules.

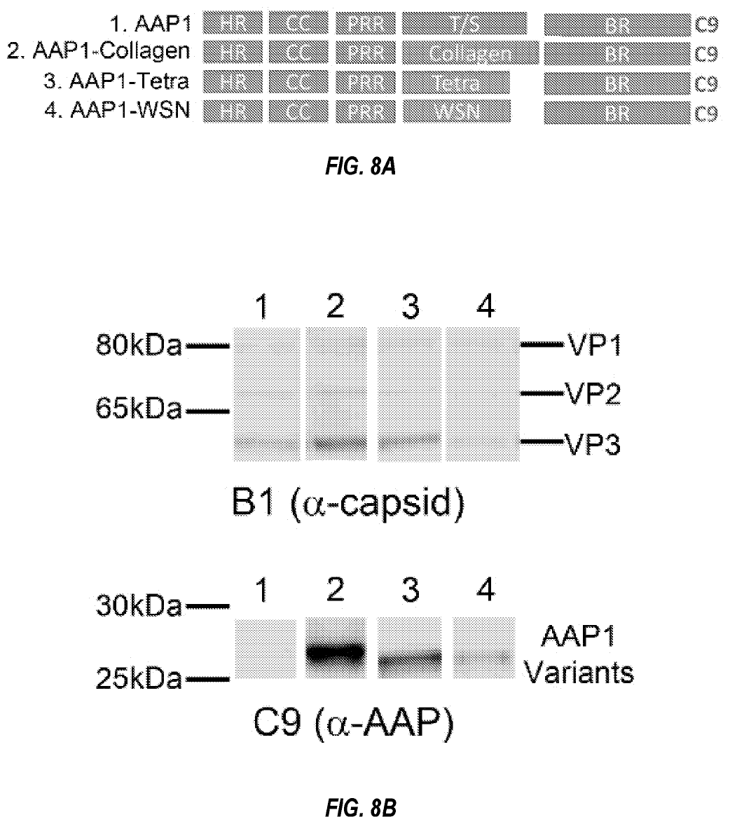

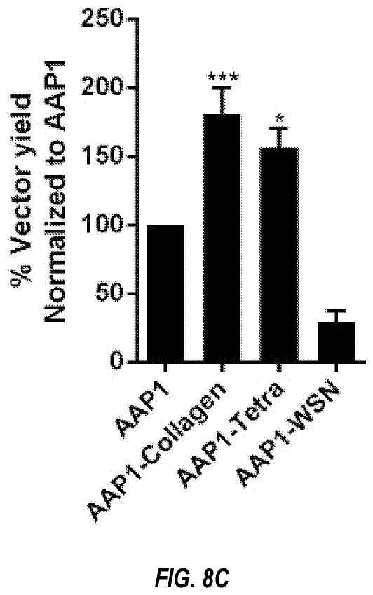

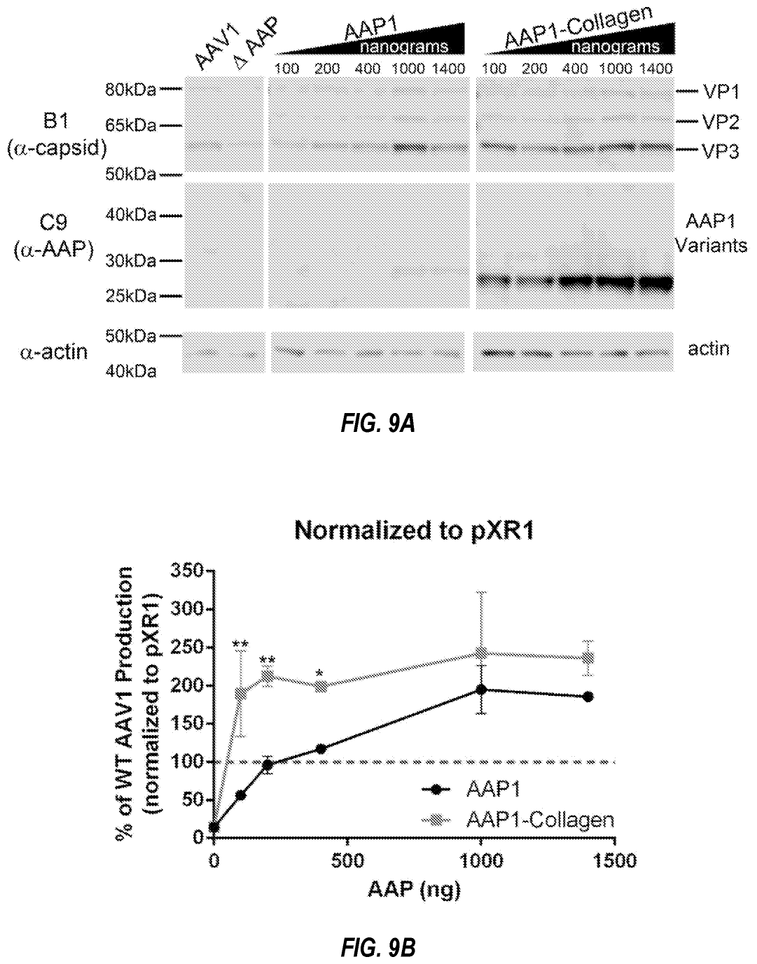

Engineering AAP for increased stability and vector production. We hypothesized that the T/S rich region of AAP is the most amenable to manipulation for imparting novel/improved functionality. By introducing EGFP in place of T/S, steady state AAP levels were likely increased probably due to increased protein stability (FIG. 2B). Thus, we attempted to further increase the stability of AAP by replacing the T/S region with oligomerization domains. We picked 3 different oligomerization domains including Collagen, which forms trimers, Influenza A/WSN/1933 hemagglutinin (HA), which is a coiled-coil trimerization domain (WSN) and a synthetic tetramerization domain (Tetra) to replace the T/S of AAP1 (FIG. 8A). Steady state AAP protein levels were increase among all the AAP variants. The increase was most pronounced in AAP1-Collagen and AAP1-Tetra, while AAP1-WSN shows a moderate increase in AAP level (FIG. 8B, Table 6). VP levels also showed an increase in case of AAP1-Collagen, remained the same in AAP1-Tetra and were slightly decreased in AAP1-WSN (FIG. 8B, Table 6). Both AAP1-Collagen and AAP1-Tetra increased rAAV vector yield at 150% and 200% compared to wildtype AAP1 respectively (FIG. 8C, Table 6). AAP1-WSN mediated vector yield is 50% less than wildtype AAP1 suggesting a potential functional incompatibility with AAP1-WSN, despite increased stability (FIG. 8C, Table 6). To further evaluate the impact of engineered AAPs on vector yield, we attempted to establish a dose-response relationship for natural and engineered AAP constructs in supporting rAAV vector production by varying the amount of AAP DNA during transfection. As expected, AAP levels increased proportionally to the amount of transfected cDNA. AAP1-Collagen shows a significant increase in AAP levels compared to wildtype AAP1 (FIG. 9A). VP levels were also increased proportional to the amount of AAP DNA, with peak titers observed at 1000 ng of AAP plasmid transfected. However, it is important to note that titers declined at higher levels potentially due to toxicity of AAP over-expression (FIG. 9A).

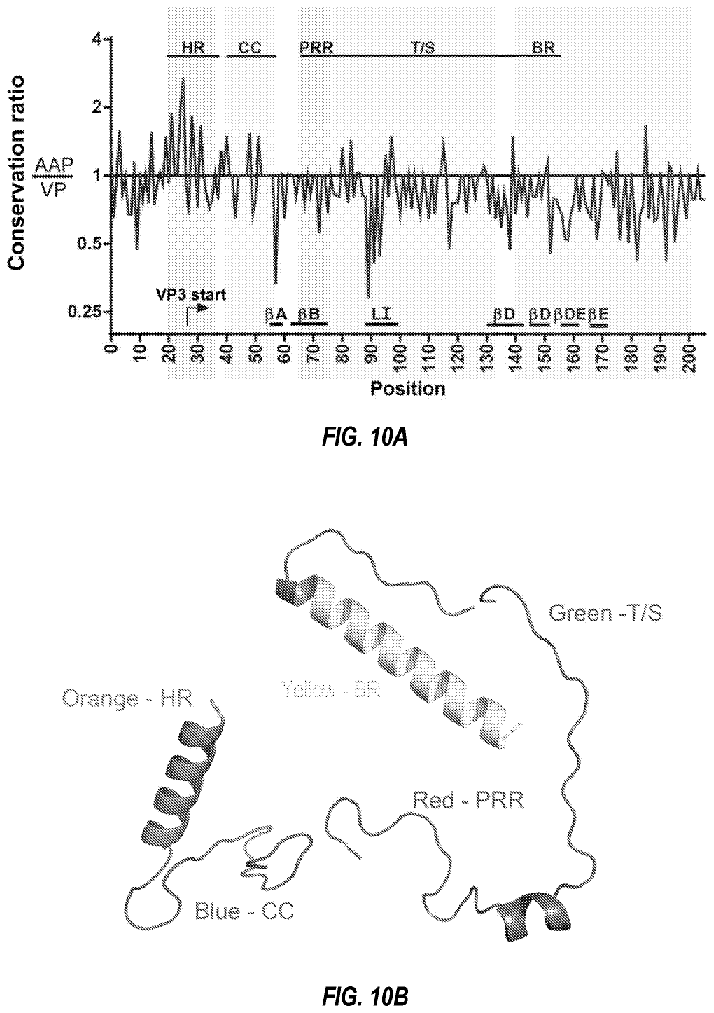

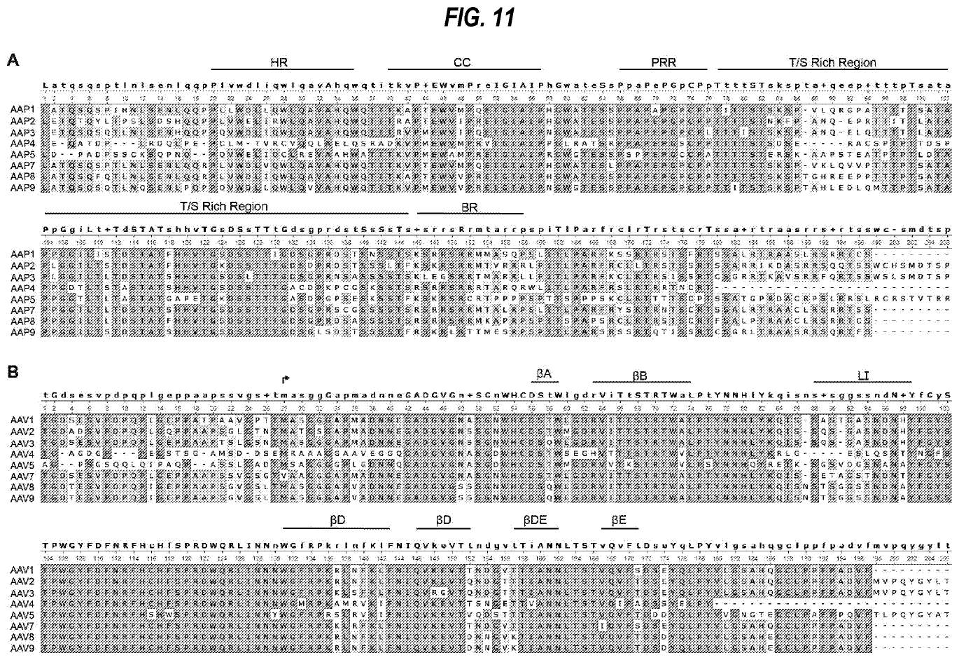

Adeno-associated virus (AAV) encodes different proteins within its 4.7 kb genome using alternative splicing, alternative start codons, and overlapping reading frames. AAP is expressed from an alternative reading frame overlapping the VP2/3 sequences. We attempted to further understand our observations by modelling the relative conservation of AAP and VP overlapping regions in the Cap gene. Briefly, we plotted the ratio (AAP/VP) of the conservation scores such that a ratio >1 denotes higher AAP conservation and a ratio <1 implies higher VP conservation. Most regions show a preference for VP over AAP, with values <1 (FIG. 10A). For instance, the sequences forming the beta strand regions that form the jelly roll structure of VP and the highly conserved loop I have AAP/VP conservation ratios ranging from 0.3-0.5 due to a higher level of VP sequence conservation. These regions correspond to the non-essential T/S rich and PRR linker domains of AAP, respectively. Thus, in these regions VP function is favored over that of AAP (FIG. 10A). In contrast, the HR and CC region show a conservation ratio >1 indicating a preference for AAP function (FIG. 10A). In corollary, this region corresponds to the VP2 N-terminal domain, which has been shown to be non-essential for AAV capsid infectivity and essentially serves as a linker between the unique VP1 N-terminal phospholipase A2 (PLA2) domain and VP3. Further, homology modeling of different AAP1E modules shows that HR is the only region that has a strong secondary structure requirement in the form of an alpha helix, while all other domains are either forming undefined loop structures or unable to be modeled (FIG. 10B; FIG. 11).

The latter theoretical observations corroborate our functional characterization of AAP. Indeed, functional analysis showed that the T/S region is dispensable and can be replaced by exogenous sequences. All of our T/S deletion or replacement constructs had higher steady state levels than the WT AAP1.

Similar to the T/S, the PRR does not have any assigned function. Deletion of the PRR and T/S together impairs capsid assembly. However, deletion of the PRR in AAP1E retains 60% of capsid assembly compared to wildtype. Replacement with the PRR from AAP5 further rescues assembly to 80%. These data suggest that the PRR plays a relatively minor role in capsid assembly and serotype specificity, but may act as a linker module that physically separates the critical HR and CC from the T/S region. Unlike the PRR and T/S, multiple studies have shown that the BR contains an important NLS/NoLS signal that is responsible for AAP localization and subsequent translocation of the capsid to the assembly site. We tested whether other AAP BR regions can functionally replace AAP1BR for capsid assembly. In our studies, only AAP4BR is able to support AAV1 capsid assembly function. As the whole AAP4 protein is unable to rescue AAP1 capsid assembly function, our data clearly demonstrate that serotype specificity is independent of the BR. We further corroborate that BR is solely acting as a NoLS in the context of AAP1 by replacing the AAP1 BR with other heterologous NLS/NoLS. Among all the NLS/NoLS tested, AP3D1 shows the best nucleolar localization and completely supports capsid assembly. The localization pattern and percent rescue of capsid assembly are highly correlated, where increased nucleolar localization indicates greater restoration of capsid assembly for AAV1. Different BR sequences have been reported for different AAPs supporting the notion that AAP had functional, rather structural evolutionary constraint in this region compared to VP.

The hydrophobic region (HR) and the conserved core (CC) are the functional domains for AAV capsid assembly and the determinants of serotype specificity. Deleting the HR or CC led to inability to pull down VP or support capsid assembly. Replacement of HR and CC from AAP5 rescued interaction with VP; however, only the 5CC replacement was able to restore capsid assembly (to 50%). As there are only two residues different between 1CC and 5CC (T44M and Q50R), it is not surprising that 5CC replacement had little effect on AAV1 capsid assembly. However, the ability of 5HR to pull down AAV1 VP was unexpected, as there are 10 differences between the two serotypes. Furthermore, binding of 5HR is not sufficient for function, as capsid assembly was defective. The HR has been predicted by homology modeling to be form an alpha-helix (FIG. 10B). Since amphiphilic helices are often found in oligomerization domains, the data support the previous finding that AAP potentially forms an oligomer. Based on our findings, we speculate that AAP interaction with the capsid is bipartite, where CC binds to a VP structural domain that is conserved among various serotypes (e.g., beta strand), and HR either binds another site or oligomerizes to facilitate formation of VP oligomer. Accordingly, capsid assembly only occurs when the two domains act together in a manner similar to a “lock and key” mechanism where CC is the backbone and HR is the gear of the key which confer serotype specificity. Alternatively, AAP interaction with VP is required to be transient whereas control release of AAP and VP interaction is required for completing capsid assembly.

Using our new knowledge of AAP structure and function, we engineered additional properties onto AAP by replacing the non-essential T/S linker region. The fluorescently traceable AAP1E, which retains the same function as the wildtype counterpart can potentially be utilized for real time, live cell imaging to study intracellular trafficking of AAP and capsid assembly events. Further, we engineered an AAP1-Collagen construct that shows improved stability and by providing this eAAP in trans, we observe a 200% increase in vector yield compared to the wildtype counterpart. Such engineered, hyper-stable AAPs could also be utilized to solve the structure of this intriguing protein as is or in complex with AAV capsid proteins. These latter observations suggest that with careful dissection of the mechanisms involving AAP biology, we can develop strategies to improve the efficiency of rAAV packaging and rAAV vector yield.

Cells, viruses and antibodies. HEK293 cells were maintained in Dulbecco's modified Eagle's medium (DMEM) supplemented with 10% fetal bovine serum (FBS) (ThermoFisher, Waltham, Mass.), 100 units/ml of penicillin and 10 μg/ml of streptomycin (P/S) (ThermoFisher, Waltham, Mass.) in 5% CO₂ at 37° C. Hybridoma supernatant of anti-AAV monoclonal antibodies B1 and A20 were produced in house and have been described earlier (ref). Mouse anti-Rhodopsin [ID4] (ab5417) and mouse anti-actin antibodies (ab3280) were purchased from Abcam (Cambridge, United Kingdom). Mouse anti-CD23 antibody [D-6] (sc-17826) was purchased from Santa Cruz Biotechnology (Santa Cruz, Calif.).

Homology modeling of AAP. Amino acid sequences of AAP1-AAP9 were used in structural prediction using SWISS-MODEL (swissmodel.expasy.org). The templates used for modelling AAP is Arabidopsis G protein coupled receptor 2, GCR2 (PDB ID: 3t33) for the HR and CC regions. The sequence identity of the modelling region is 12.20% and the global model quality estimation (GMQE) and QMEAN Z-score are 0.08 and -1.42. Different domains of AAP were also predicted individually and yield similar result. The HR domain can also be modeled with bacterial RNase ligase (PDB ID: 4xru) and E. coli. topoisomerase (PDB ID: 1yua) with QMEAN Z-score at −1.81 and −2.08 respectively.

Bioinformatic analysis. Sequences from AAP and VP (only the residues corresponding to those in AAP) were aligned using MUSCLE followed by manual adjustment. Alignments were done such that residues and gaps directly correspond in AAP and VP. Percent identity and similarity of AAPs from other serotypes compared to AAP1 were calculated using the Sequence Manipulation Suite using the following groups for similarity: GAVLI, FYW, CM, ST, KRH, DENQ, P. Amino acid conservation scores at each position of AAP and VP were calculated using the prediction tool developed by Capra and Singh. Analysis was run using the property entropy scoring method, sequence weighting, the BLOSUM62 background and scoring matrix and a window size of 0.

Generation of different AAP constructs. All AAP construct were cloned into pCDNA3.1 using EcoRI and NotI site. Chimera constructs were cloned using either overlapping PCR or Gibson Assembly (NEBuilder HiFi, New England Biolabs, Ipswich, Mass.). Detail designs of each construct is illustrated in each figure.

AAP and capsid expression of different AAP constructs by western blot. HEK293 cells at 60%-70% confluency on a 6-well plate were transfected with pXX680 (600 ng), pTR-CBA-Luc (400 ng), pXR-AAV1-no AAP (600 ng) and pCDNA3.1 AAP constructs (400 ng) using polyethylenimine (PEI) as the transfection reagent. At three days post transfection, cell pellets were washed 2 times with 1×DPBS and lysed with 200 μl of 1×passive lysis buffer (Promega, Madison, Wis.) for 30 min on ice with Halt Protease inhibitor (ThermoFisher, Waltham, Mass.). Supernatants were collected after centrifugation at 13,000 g for 5 min at 4° C. The sample were prepared for western blotting in 1×LDS loading dye (ThermoFisher, Waltham, Mass.) and 100 mM DTT, then boiled at 95° C. for 5 min and loaded on a NuPAGE 4-12% Bis-Tris SDS-page gel (ThermoFisher, Waltham, Mass.). The protein bands were transferred to nitrocellullose membrane (ThermoFisher, Waltham, Mass.) using a semi-dry Xcell Surelock module (ThermoFisher, Waltham, Mass.). VP, AAP and actin proteins were detected by B1 hybridoma supernatant at 1:50 dilution, mouse α-Rhodopsin [ID4] antibody (ab5417) at 1:2000 dilution and α-actin antibodies (ab3280) at 1:1000 dilution respectively; goat α-mouse-HRP at 1:10000 dilution was used as the secondary antibody. Chemiluminescence reaction was initiated with enhanced chemiluminescence (ECL) substrate (SuperSignal West Femto Maximum Sensitivity, ThermoFisher, Waltham, Mass.) and the membrane is developed on an AI600RGB system (Amersham Biosciences, Little Chalfont, United Kingdom).

AAP dependent capsid assembly/vector production by quantitative PCR. HEK293 cells at 60%-70% confluency on a 6-well plate were transfected with pXX680 (600 ng), pTR-CBA-Luc (400 ng), pXR-AAV1-no AAP (600 ng) and pCDNA3.1 AAP constructs (400 ng) using polyethylenimine (PEI). Transfection media was replaced with fresh media after 24 h and supernatant was harvested at five day post transfection. Supernatants were collected after centrifugation at 13,000 g for 2 min. The supernatants were used directly for standard qPCR analysis to determine vector yield and for transduction assay. Subsequent steps involving harvesting of recombinant AAV vectors and downstream purification were carried out as described earlier. In brief, supernatant were treated with DNase (90 μg/ml) for 1 h at 37° C. DNase was inactivated by the addition of EDTA (13.2 mM) followed by proteinase K (0.53 mg/ml) digestion for 2 h at 55° C. Recombinant AAV vector titers were determined by quantitative PCR (qPCR) with primers that amplify AAV2 inverted terminal repeat (ITR) regions, 5′-AACATGCTACGCAGAGAGGGAGTGG-3′ (SEQ ID NO:17), 5′-CATGAGACAAGGAACCCCTAGTGATGGAG-3′ (SEQ ID NO:18). Relative vector yields from different AAP constructs were normalized to wildtype AAP1 unless specifically indicated in figure legend.

In vitro AAV transduction assays. AAV vectors produced with different AAP constructs packaging ssCBA-Luc transgenes were pre-diluted in DMEM+5% FBS+P/S. 50 microliters of recombinant AAV vectors (1,000-10,000 vg/cell) were mixed with 50 μl of 5×10⁴ HEK293 cells and added to tissue culture treated, black, glass bottom 96 well plates (Corning, Corning, N.Y.). The plates were incubated in 5% CO₂ at 37° C. for 48 h. Cells were then lysed with 25 μl of 1× passive lysis buffer (Promega, Madison, Wis.) for 30 min at RT. Luciferase activity was measured on a Victor 3 multilabel plate reader (Perkin Elmer, Waltham, Mass.) immediately after addition of 25 μl of luciferin (Promega, Madison, Wis.). All readouts were normalized to wild type AAP1 controls.

Immunofluorescence and confocal microscopy. HEK293 cells were seeded on a 12 mm poly-lysine treated glass coverslip (GG-12-1.5-PDL, NeoVitro, Vancouver, Wash.) in a 24 well plate. Cells were transfected at 60-70% confluency with pCDNA3.1 AAP alone (250 ng) using PEI as the transfection reagent. At 2 days post-transfection, cells were fixed with 4% PFA in PBS for 15 min and permeabilized with 0.1% Triton X-100 in PBS for 10 min. Nucleolus was stained using α-C23 [D-6] antibody followed by goat α-mouse IgG H+L AlexaFluor 594 (ThermoFisher, Waltham Mass.). Nucleus was stained with DAPI (ThermoFisher, Waltham, Mass.). Coverslip were mounted onto microscope slide using ProLong Diamond mountant (Invitrogen, Carlsbad, Calif.). Fluorescence images were taken by Zeiss LSM 710 Spectral Confocal Laser Scanning Microscope at the UNC Microscopy Service Laboratory (MSL).

Immunoprecipitation assays. HEK293 cells at 60%-70% confluency on a 10 cm plate were transfected with pXX680 (3000 ng), pTR-CBA-Luc (2500 ng), pXR-AAV1-no AAP (3000 ng) and pCDNA3.1 AAP constructs (7500 ng) using polyethylenimine (PEI) as the transfection reagent. At three days post transfection, cells were harvested from the plate in cold 1×DPBS followed by two washes in 1×DPBs. Pellets were resuspended in 400 uL of Buffer D (20 mM Hepes/KOH pH 7.9, 25% glycerol, 0.1 M KCl, 0.2 mM EDTA) and lysed on ice for 30 minutes with Halt protease inhibitor (ThermoFisher, Waltham Mass.). Lysates were spun at 13,000×g for two minutes. 5% of the supernatant was retained as input and prepared in 1×LDS buffer and 100 mM DTT. 10 μl, of protein G beads (washed three times in Buffer D) was added to the remaining lysate, then placed on a rotator at 4° C. for 2 hours. Following, beads were washed twice for thirty minutes each in Buffer D, followed by resuspension in 1×LDS buffer and 100 mM DTT. Samples were denatured at 95° C. for 5 minutes, then loaded onto a precast 10% Bis-tris gel (ThermoFisher, Waltham, Mass.) and run in MOPS-SDS buffer. Protein was transferred to a 0.45 micron nitrocellulose membrane (ThermoFisher, Waltham, Mass.) in a wet transfer apparatus. AAP was detected using α-human-HRP at a 1:10000 dilution and visualized after reaction with Femto western blot substrate (ThermoFisher, Waltham, Mass.) on an Amersham AI600RGB system (Amersham Biosciences, Little Chalfont, United Kingdom). Membranes were incubated in 30% peroxide for 30 minutes at room temperature, then re-blocked. VP, AAP and actin proteins were detected by B1 hybridoma supernatant at 1:50 dilution and α-actin antibody (ab3280) at 1:2000 dilution, respectively; goat anti α-mouse-HRP at a 1:20000 dilution was used as the secondary antibody. Membranes were again visualized as described above.

### Example 2. Mapping and Engineering Functional Domains of the Assembly Activating Protein (AAP) of Adeno-Associated Viruses (Abstract)

Adeno-associated viruses (AAV) encode a unique assembly activating protein (AAP) within their genome that is essential for capsid assembly. Studies to date have focused on establishing the role (or lack thereof) of AAP as a chaperone that mediates stability, nucleolar transport, and assembly of AAV capsid proteins. Here, we map structure-function correlates of AAP based on secondary structure and bioinformatics, followed by deletion and substitutional analysis of specific domains, namely, the hydrophobic N-terminal domain (HR), conserved core (CC), proline-rich region (PRR), threonine/serine rich region (T/S) and basic region (BR). First, we establish that the hydrophobic region (HR) and the conserved core (CC) in the AAP N-terminus are the sole determinants for viral protein (VP) recognition. However, VP recognition alone is not sufficient for capsid assembly or conferring serotype specificity. Enhancing the hydrophobicity and alpha-helical nature of the N-terminal AAP region through amino acid substitutions enabled assembly of previously unrecognized VPs into capsids. Interestingly, the adjacent PRR and T/S regions are flexible linker domains that can either be deleted completely or replaced by heterologous functional domains that enable ancillary functions such as fluorescence imaging and precise control over oligomerization. We also demonstrate that the C-terminal BR domains can be substituted with heterologous nuclear and nucleolar localization sequences that display varying efficiency or with IgG Fc domains for VP complexation and structural analysis. The newly engineered AAPs (eAAP) are more stable and require only about 20% of the original AAP sequence for efficiently supporting AAV capsid assembly. Our study sheds light on the structure-function correlates of AAP and provides multiple examples of engineered AAP that might prove useful for understanding and controlling AAV capsid assembly.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. The invention is defined by the following claims, with equivalents of the claims to be included therein.

TABLE 1

| AAV | GenBank |
|---|---|
| Serotypes/ | Accession |
| Isolates | Number |
| Clonal Isolates | |
| Avian AAV ATCC | AY186198, |
| VR-865 | AY629583, |
| | NC_004828 |
| Avian AAV strain | NC_006263, |
| DA-1 | AY629583 |
| Bovine AAV | NC_005889, |
| | AY-88617 |
| AAV4 | NC_001829 |
| AAV5 | AY18065, |
| | AF085716 |
| Rh34 | AY243001 |
| Rh33 | AY243002 |
| Rh32 | AY243003 |
| Clade A | |
| AAV1 | NC_002077, |
| | AF063497 |
| AAV6 | NC_001862 |
| Hu.48 | AY530611 |
| Hu 43 | AY530606 |
| Hu 44 | AY530607 |
| Hu 46 | AY530609 |
| Clade B | |
| Hu19 | AY530584 |
| Hu20 | AY530586 |
| Hu23 | AY530589 |
| Hu22 | AY530588 |
| Hu24 | AY530590 |
| Hu21 | AY530587 |
| Hu27 | AY530592 |
| Hu28 | AY530593 |
| Hu29 | AY530594 |
| Hu63 | AY530624 |
| Hu64 | AY530625 |
| Hu13 | AY530578 |
| Hu56 | AY530618 |
| Hu57 | AY530619 |
| Hu49 | AY530612 |
| Hu58 | AY530620 |
| Hu34 | AY530598 |
| Hu35 | AY530599 |
| AAV2 | NC_001401 |
| Hu45 | AY530608 |
| Hu47 | AY530610 |
| Hu51 | AY530613 |
| Hu52 | AY530614 |
| Hu T41 | AY695378 |
| Hu S17 | AY695376 |
| Hu T88 | AY695375 |
| Hu T71 | AY695374 |
| Hu T70 | AY695373 |
| Hu T40 | AY695372 |
| Hu T32 | AY695371 |
| Hu T17 | AY695370 |
| Hu LG15 | AY695377 |
| Clade C | |
| AAV 3 | NC_001729 |
| AAV 3B | NC_001863 |
| Hu9 | AY530629 |
| Hu10 | AY530576 |
| Hu11 | AY530577 |
| Hu53 | AY530615 |
| Hu55 | AY530617 |
| Hu54 | AY530616 |
| Hu7 | AY530628 |
| Hu18 | AY530583 |
| Hu15 | AY530580 |
| Hu16 | AY530581 |
| Hu25 | AY530591 |
| Hu60 | AY530622 |
| Ch5 | AY243021 |
| Hu3 | AY530595 |
| Hu1 | AY530575 |
| Hu4 | AY530602 |
| Hu2 | AY530585 |
| Hu61 | AY530623 |
| Clade D | |
| Rh62 | AY530573 |
| Rh48 | AY530561 |
| Rh54 | AY530567 |
| Rh55 | AY530568 |
| Cy2 | AY243020 |
| AAV7 | AF513851 |
| Rh35 | AY243000 |
| Rh37 | AY242998 |
| Rh36 | AY242999 |
| Cy6 | AY243016 |
| Cy4 | AY243018 |
| Cy3 | AY243019 |
| Cy5 | AY243017 |
| Rh13 | AY243013 |
| Clade E | |
| Rh38 | AY530558 |
| Hu66 | AY530626 |
| Hu42 | AY530605 |
| Hu67 | AY530627 |
| Hu40 | AY530603 |
| Hu41 | AY530604 |
| Hu37 | AY530600 |
| Rh40 | AY530559 |
| Rh2 | AY243007 |
| Bb1 | AY243023 |
| Bb2 | AY243022 |
| Rh10 | AY243015 |
| Hu17 | AY530582 |
| Hu6 | AY530621 |
| Rh25 | AY530557 |
| Pi2 | AY530554 |
| Pi1 | AY530553 |
| Pi3 | AY530555 |
| Rh57 | AY530569 |
| Rh50 | AY530563 |
| Rh49 | AY530562 |
| Hu39 | AY530601 |
| Rh58 | AY530570 |
| Rh61 | AY530572 |
| Rh52 | AY530565 |
| Rh53 | AY530566 |
| Rh51 | AY530564 |
| Rh64 | AY530574 |
| Rh43 | AY530560 |
| AAV8 | AF513852 |
| Rh8 | AY242997 |
| Rh1 | AY530556 |
| Clade F | |
| AAV9 (Hu14) | AY530579 |
| Hu31 | AY530596 |
| Hu32 | AY530597 |

TABLE 2
Amino acid residues and abbreviations

| | Abbreviation | |
|---|---|---|
| | Three-Letter | One-Letter |
| Amino Acid Residue | Code | Code |
| Alanine | Ala | A |
| Arginine | Arg | R |
| Asparagine | Asn | N |
| Aspartic acid (Aspartate) | Asp | D |
| Cysteine | Cys | C |
| Glutamine | Gln | Q |
| Glutamic acid (Glutamate) | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Praline | Pro | P |
| Serine | Ser | S |
| Threonine | Thr | T |
| Tryptophan | Trp | W |
| Tyrosine | Tyr | Y |
| Valine | Val | V |

TABLE 3

| Serotype | Position 1 | Position 2 |
|---|---|---|
| AAV1 | A263X | T265X |
| AAV2 | Q263X | −265X |
| AAV3a | Q263X | −265X |
| AAV3b | Q263X | −265X |
| AAV4 | S257X | −259X |
| AAV5 | G253X | V255X |
| AAV6 | A263X | T265X |
| AAV7 | E264X | A266X |
| AAV8 | G264X | S266X |
| AAV9 | S263X | S265X |

Where, (X)→ mutation to any amino acid
(−)→ insertion of any amino acid
Note:
Position 2 inserts are indicated by the site of insertion

TABLE 4

| Modified Amino Acid Residue | |
|---|---|
| Amino Acid Residue Derivatives | Abbreviation |
| 2-Aminoadipic acid | Aad |
| 3-Aminoadipic acid | bAad |
| beta-Alanine, beta-Aminoproprionic acid | bAla |
| 2-Aminobutyric acid | Abu |
| 4-Aminobutyric acid, Piperidinic acid | 4Abu |
| 6-Aminocaproic acid | Acp |
| 2-Aminoheptanoic acid | Ahe |
| 2-Aminoisobutyric acid | Aib |
| 3-Aminoisobutyric acid | bAib |
| 2-Aminopimelic acid | Apm |
| t-butylalanine | t-BuA |
| Citrulline | Cit |
| Cyclohexylalanine | Cha |
| 2,4-Diaminobutyric acid | Dbu |
| Desmosine | Des |
| 2,2′-Diaminopimelic acid | Dpm |
| 2,3-Diaminoproprionic acid | Dpr |
| N-Ethylglycine | EtGly |
| N-Ethylasparagine | EtAsn |
| Homoarginine | hArg |
| Homocysteine | hCys |
| Homoserine | hSer |
| Hydroxylysine | Hyl |
| Allo-Hydroxylysine | aHyl |
| 3-Hydroxyproline | 3Hyp |
| 4-Hydroxyproline | 4Hyp |
| Isodesmosine | Ide |
| allo-Isoleucine | alle |
| Methionine sulfoxide | MSO |
| N-Methylglycine, sarcosine | MeGly |
| N-Methylisoleucine | Melle |
| 6-N-Methyllysine | MeLys |
| N-Methylvaline | MeVal |
| 2-Naphthylalanine | 2-Nal |
| Norvaline | Nva |
| Norleucine | Nle |
| Ornithine | Orn |
| 4-Chlorophenylalanine | Phe(4-Cl) |
| 2-Fluorophenylalanine | Phe(2-F) |
| 3 -Fluorophenylalanine | Phe(3-F) |
| 4-Fluorophenylalanine | Phe(4-F) |
| Phenylglycine | Phg |
| Beta-2-thienylalanine | Thi |

TABLE 5
Amino acid sequences of different T/S and BR module
replacement constructs

| | Amino acid Sequences |
|---|---|
| T/S replacements | |
| Silk (14) | GAGAGAGQGAGAGAGQGAAAGAGAGAGQT (SEQ ID NO: 3) |
| Silk E1 (14) | GAGAGQGAGAGAGQGAAAGAGAGAGQGAGAGAGQGAGAGAGQG |
| | AAGAGAGAGQT (SEQ ID NO: 4) |
| Full MLD (15) | PTPVTAIGPPTTAIQEPPSRIVPTPTSPAIAPPTETMAPPVRDP |
| | VPGKPTVTIRTRGAIIQTPTLGPIQPTRVSEAGTTVPGQIRPTL |
| | TIPGYVEPTAVITPPTTTTKKPRVSTPKPATPSTDSSTTTTRRP |
| | TKKPRTPRPVPRVTTK (SEQ ID NO: 5) |
| Partial MLD (15) | TLTIPGYVEPTAVITPPTTTTKKPRVSTPKPATPSTDSSTTTTR |
| | RPTKKPRTPRPVPRVTT (SEQ ID NO: 6) |
| Collagen (21) | GSSGVRLWATRQAMLGQVHEVPEGWLIFVAEQEELYVRVQNGFR |
| | KVQLEARTPLPR (SEQ ID NO: 7) |
| Tetramer (22) | AEIEQAKKEIAYLIKKAKEEILEEIKKAKQEIA (SEQ ID |
| | NO: 8) |
| WSN | KRMENLNKKVDDGFLDIWTYNAELLVLLENERTLDFHDLNVKNL |
| | YEKVKSQLK (SEQ ID NO: 19) |
| BR replacements | |
| Rpp29 (16) | RHKRKEKKKKAKGLSARQRRELR (SEQ ID NO: 10) |
| AP3D1 (17) | RRHRQKLEKDKRRKKRKEKEERTKGKKKSKK (SEQ ID NO: |
| | 11) |
| SV40 (18, 19) | KRTADGSEFESPKKKRKVE (SEQ ID NO: 12) |
| HIV Rev (20) | RQARRNRRRRWRERQR (SEQ ID NO: 13) |
| 4BR | STSRSRRSRRRTARQRWLITLPARFRSLRTRRTNCRT |
| | (SEQ ID NO: 20) |
| 5BR | STFKSKRSRCRTPPPPSPTTSPPPSKCLRTTTTSCPTSSATG |
| | PRDACRPSLRRSLRCRSTVTRR (SEQ ID NO: 21) |
| 9BR | STFRSKRLRTTMESRPSPITLPARSRSSRTQTISSRTCSGRL |
| | TRAASRRSQRTFS (SEQ ID NO: 22) |

TABLE 6
Summary of biological properties of T/S and N-terminal constructs.
WT represents similar properties, “−“ represents reduced
levels and “+” represents increased levels comparing to AAP1
wildtype. The number of “+” and “−“ are proportional to the increase or
decrease in expression levels or titer.

| | AAP | VP | Vector | Interaction |
|---|---|---|---|---|
| T/S constructs | level | Level | yield | w/VP |
| AAP1 wildtype | WT | WT | WT | Yes |
| TL1 | ++ | WT | + | n/a |
| TL2 | WT | − | −−− | n/a |
| TL3 | WT | − | −−− | n/a |
| TL4 | − | − | −−− | n/a |
| Silk | ++ | WT | + | n/a |
| SilkE1 | + | WT | + | n/a |
| Full MID | WT | − | WT | n/a |
| Partial MLD | +++ | WT | + | n/a |
| AAP1E | ++++ | + | WT | Yes |
| AAP1-Collagen (21) | ++++++ | ++ | +++ | n/a |
| AAP1-Tetra (22) | +++++ | + | + | n/a |
| AAP1-WSN | ++ | WT | − | n/a |
| N-terminal | AAP | VP | Vector | Interaction |
| constructs | level | Level | yield | w/VP |
| AAP1 wildtype | WT | WT | WT | Yes |
| AAP1E | ++++ | + | WT | Yes |
| AAP5E | ++++ | −− | −−− | Yes |
| ΔHR | ++++++ | −− | −−− | No |
| 5HR | ++++ | −− | −−− | Yes |
| ΔCC | ++++ | −− | −−− | No |
| 5CC | + | − | −− | Yes |
| ΔPRR | ++++ | WT | −− | Yes |
| 5PRR | WT | WT | WT | Yes |

TABLE 7
Summary of biological properties of BR constructs. WT represents
similar properties, “−“ represents reduced level and “+”
represents increased levels comparing to AAP1 wildtype.
The number of “+” and “−“ are proportional to the increase or decrease in
expression level or titer.

| BR constructs | AAP level | VP Level | Vector yield | AAP localization |
|---|---|---|---|---|
| AAP1 wildtype | WT | WT | WT | Nucleolus |
| DBR | ++++ | WT | −− | Cytoplasmic |
| 4BR | +++++ | + | + | Nucleolus |
| 5BR | +++++ | WT | − | Nucleus |
| 9BR | +++ | − | −− | Nucleus |
| Rpp29 | ++++ | − | − | Nucleus/Nucleolus |
| AP3D1 | ++++ | WT | WT | Nucleolus |
| SV40 | ++++ | + | −− | Nucleus |
| HIV Rev | ++++ | − | − | Nucleolus |

SEQUENCES:
EGFP:
(SEQ ID NO: 2)
MVSKGEELFTGVVPILVELDGDVNGHKFSVSGEGEGDATYGKLTLKFICTTGKLPVPWPTLV
TTLTYGVQCFSRYPDHMKQHDFFKSAMPEGYVQERTIFFKDDGNYKTRAEVKFEGDTLVNR
IELKGIDFKEDGNILGHKLEYNYNSFINVYIMADKQKNGIKVNFKIRHNIEDGSVQLADHYQQ
NTPIGDGPVLLPDNHYLSTQSALSKDPNEKRDHMVLLEFVTAAGITLGMDELYK
Silk:
(SEQ ID NO: 3)
GAGAGAGQGAGAGAGQGAAAGAGAGAGQT
pMLD:
(SEQ ID NO: 6)
TLTIPGYVEPTAVITPPTTTTKKPRVSTPKPATPSTDSSTTTTRRPTKKPRTPRPVPRVTT
Collagen:
(SEQ ID NO: 7)
GSSGVRLWATRQAMLGQVITBVPEGWLIFVAEQEELYVRVQNGFRKVQLEARTPLPR
Tetramer:
(SEQ ID NO: 8)
AEIEQAKKEIAYLIKKAKEEILEEIKKAKQEIA
AAP sequences of AAV serotypes 1-9 with proline rich region
>AAP1
(SEQ ID NO: 23)
LATQSQSPIHNLSENLQQPPLLWDLLQWLQAVAHQWQTITKAPTEWVMPQEIGIAIPHGWAT
ESSPPAPAPGPCPPTITTSTSKSPVLQRGPATTTTTSATAPPGGILISTDSTATFHHVTGSD
SSTTIGDSGPRDSTSNSSTSKSRRSRRMMASQPSLITLPARFKSSRTRSTSFRTSSALRTRA
ASLRSRRTCS
>AAP2
(SEQ ID NO: 24)
LETQTQYLTPSLSDSHQQPPLVWELIRWLQAVAHQWQTITRAPTEWVIPREIGIAIPHGWAT
ESSPPAPEPGPCPPTTTTSTNKFPANQEPRTTITTLATAPLGGILTSTDSTATFHHVTGKDS
STTTGDSDPRDSTSSSLTFKSKRSRRMTVRRRLPITLPARFRCLLTRSTSSRTSSARRIKDA
SRRSQQTSSWCHSMDTSP
>AAP3
(SEQ ID NO: 25)
LETQSQSQTLNLSENHQQPPQVWDLIQWLQAVAHQWQTITRVPMEWVIPQEIGIAIPNGWAT
ESSPPAPEPGPCPLTTTISTSKSPANQELQTTTTTLATAPLGGILTLTDSTATSHHVTGSDS
LTTTGDSGPRNSASSSSTSKLEGSRRTMARRLLPITLPARFKCLRTRSISSRTCSGRRTKAV
SRRFQRTSSWSLSMDTSP
>AAP4
(SEQ ID NO: 26)
LEQATDPLRDQLPEPCLMTVRCVQQLAELQSRADKVPMEWVMPRVIGIAIPPGLRATSRPPA
PEPGSCPPTTTTSTSDSERACSPTPTTDSPPPGDTLTSTASTATSHHVTGSDSSTTTGACDP
KPCGSKSSTSRSRRSRRRTARQRWLITLPARFRSLRTRRTNCRT
>AAP5
(SEQ ID NO: 27)
LDPADPSSCKSQPNQPQVWELIQCLREVAAHWATITKVPMEWAMPREIGIAIPRGWGTESSP
SPPEPGCCPATTTTSTERSKAAPSTEATPTPTLDTAPPGGTLTLTASTATGAPETGKDSSTT
TGASDPGPSESKSSTFKSKRSRCRTPPPPSPTTSPPPSKCLRTTTTSCPTSSATGPRDACRP
SLRRSLRCRSTVTRR
>AAP6
(SEQ ID NO: 28)
LATQSQSPTHNLSENLQQPPLLWDLLQWLQAVAHQWQTITKAPTEWVMPQEIGIAIPHGWAT
ESSPPAPEHGPCPPITTTSTSKSPVLQRGPATTTTTSATAPPGGILISTDSTAISHHVTGSD
SSTTIGDSGPRDSTSSSSTSKSRRSRRMMASRPSLITLPARFKSSRTRSTSCRTSSALRTRA
ASLRSRRTCS
>AAP7
(SEQ ID NO: 29)
LATQSQSPTLNLSENLQQRPLVWDLVQWLQAVAHQWQTITKVPTEWVMPQEIGIAIPHGWAT
ESLPPAPEPGPCPPTTTTSTSKSPVKLQVVPTTTPTSATAPPGGILTLTDSTATSHHVTGSD
SSTTTGDSGPRSCGSSSSTSRSRRSRRMTALRPSLITLPARFRYSRTRNTSCRTSSALRTRA
ACLRSRRTSS
>AAP8
(SEQ ID NO: 30)
LATQSQFQTLNLSENLQQRPLVWDLIQWLQAVAHQWQTITKAPTEWVVPREIGIAIPHGWAT
ESSPPAPEPGPCPPTTTTSTSKSPTGHREEPPTTTPTSATAPPGGILTLTDSTATFHHVTGS
DSSTTTGDSGPRDSASSSSTSRSRRSRRMKAPRPSPITSPAPSRCLRTRSTSCRTFSALPTR
AACLRSRRTCS
>AAP9
(SEQ ID NO: 31)
LATQSQSQTLNQSENLPQPPQVWDLLQWLQVVAHQWQTITKVPMEWVVPREIGIAIPNGWGT
ESSPPAPEPGPCPPTTITSTSKSPTAHLEDLQMTTPTSATAPPGGILTSTDSTATSHHVTGS
DSSTTTGDSGLSDSTSSSSTFRSKRLRTTMESRPSPITLPARSRSSRTQTISSRTCSGRLTR
AASRRSQRTFS
AAP Amino Acid Alignment in AAV serotypes 1-9

| | | |
|---|---|---|
| AAP1 | LATQSQSPIHNLSENLQQPPLLWDLLQWLQAVAHQWQTITKAPTEWVMPQEIGIAIPHGW | 60 |
| AAP2 | LETQTQYLTPSLSDSHQQPPLVWELIRWLQAVAHQWQTITRAPTEWVIPREIGIAIPHGW | 60 |
| AAP3 | LETQSQSQTLNLSENHQQPPQVWDLIQWLQAVAHQWQTITRVPMEWVIPQEIGIAIPNGW | 60 |
| AAP4 | LE----QATDPLRDQLPEP--CLMTVRCVQQLAELQSRADKVPMEWVMPRVIGIAIPPGL | 54 |
| AAP5 | ----LDPADPSSCKSQPNQPQVWELIQCLREVAAHWATITKVPMEWAMPREIGIAIPRGW | 56 |
| AAP6 | LATQSQSPTHNLSENLQQPPLLWDLLQWLQAVAHQWQTITKAPTEWVMPQEIGIAIPHGW | 60 |
| AAP7 | LATQSQSPTLNLSENLQQRPLVWDLVQWLQAVAHQWQTITKVPTEWVMPQEIGIAIPHGW | 60 |
| AAP8 | LATQSQFQTLNLSENLQQRPLVWDLIQWLQAVAHQWQTITKAPTEWVVPREIGIAIPHGW | 60 |
| AAP9 | LATQSQSQTLNQSENLPQPPQVWDLLQWLQVVAHQWQTITKVPMEWVVPREIGIAIPNGW | 60 |
| | .. : :: :: :* :.* **.:*: ****** * | |
| AAP1 | ATESSPPAPAPGPCPPTITTSTSKSPVLQR-GPATTTTTSATAPPGGILISTDSTATFHH | 119 |
| AAP2 | ATESSPPAPEPGPCPPTTTTSTNKFPANQ--EPRTTITTLATAPLGGILTSTDSTATFHH | 118 |
| AAP3 | ATESSPPAPEPGPCPLTTTISTSKSPANQ--ELQTTTTTLATAPLGGILTLTDSTATSHH | 118 |
| AAP4 | RATSRPPAPEPGSCPPTTTTSTSDSERAC-----SPTPTTDSPPPGDTLTSTASTATSHH | 109 |
| AAP5 | GTESSPSPPEPGCCPATTTTSTERSKAAPS-TEATPTPTLDTAPPGGTLTLTASTATGAP | 115 |
| AAP6 | ATESSPPAPEHGPCPPITTTSTSKSPVLQR-GPATTTTTSATAPPGGILISTDSTAISHH | 119 |
| AAP7 | ATESLPPAPEPGPCPPTTTTSTSKSPVKLQ-VVPTTTPTSATAPPGGILTLTDSTATSHH | 119 |
| AAP8 | ATESSPPAPEPGPCPPTTTTSTSKSPTGHREEPPTTTPTSATAPPGGILTLTDSTATFHH | 120 |
| AAP9 | GTESSPPAPEPGPCPPTTITSTSKSPTAHLEDLQMTTPTSATAPPGGILTSTDSTATSHH | 120 |
| | : * * * * ** **. * : * *. * * *** | |
| AAP1 | VTGSDSSTTIGDSGPRDSTSNSSTSKSRRSRRMMASQPSLITLPARFKSSRTRSTSFRTS | 179 |
| AAP2 | VTGKDSSTTTGDSDPRDSTSSSLTFKSKRSRRMTVRRRLPITLPARFRCLLTRSTSSRTS | 178 |
| AAP3 | VTGSDSLTTTGDSGPRNSASSSSTSKLEGSRRTMARRLLPITLPARFKCLRTRSISSRTC | 178 |
| AAP4 | VTGSDSSTTTGACDPKPCGSKSSTSRSRRSRRRTARQRWLITLPARFRSLRTRRTNCRT- | 168 |
| AAP5 | ETGKDSSTTTGASDPGPSESKSSTFKSKRSRCRTPPPPSPTTSPPPSKCLRTTTTSCPTS | 175 |
| AAP6 | VTGSDSSTTIGDSGPRDSTSSSSTSKSRRSRRMMASRPSLITLPARFKSSRTRSTSCRTS | 179 |
| AAP7 | VTGSDSSTTTGDSGPRSCGSSSSTSRSRRSRRMTALRPSLITLPARFRYSRTRNTSCRTS | 179 |
| AAP8 | VTGSDSSTTTGDSGPRDSASSSSTSRSRRSRAMKAPRPSPITSPAPSRCLRTRSTSCRTF | 180 |
| A7P9 | VTGSDSSTTTGDSGLSDSTSSSSTFRSKRLRTTMESRPSPITLPARSRSSRTQTISSRTC | 180 |
| | **.** ** * .. . *.* * : . * * * : * . * | |
| AAP1 | SALRTRAASLRSRRTCS--------- 196 (SEQ ID NO: 23) | |
| A2P2 | SARRIKDASRRSQQTSSWCHSMDTSP 204 (SEQ ID NO: 24) | |
| AAP3 | SGRRTKAVSRREQRTSSWSLSMDTSP 204 (SEQ ID NO: 25) | |
| AAP4 | -------------------------- 168 (SEQ ID NO: 26) | |
| AAP5 | SATGPRDACRPSLRRSLRCRSTVTRR 201 (SEQ ID NO: 27) | |
| AAP6 | SALRTRAASLRSRRTCS--------- 196 (SEQ ID NO: 28) | |
| AAP7 | SALRTRAACLRSRRTSS--------- 196 (SEQ ID NO: 29) | |
| AAP8 | SALPTRAACLRSRRTCS--------- 197 (SEQ ID NO: 30) | |
| A1P9 | SGRLTRAASRRSQRTFS--------- 197 (SEQ ID NO: 31) | |

AAP DNA Alignment in AAV seroptypes 1-9

| | | |
|---|---|---|
| AAP1 | CTGGCGACTCAGAGTCAGTCCCCGATCCACAACCTCTCGGAGAACCTCCAGCAACCCCCG | 60 |
| AAP2 | CTGGAGACGCAGACTCAGTACCTGACCCCCAGCCTCTCGGACAGCCACCAGCAGCCCCCT | 60 |
| AAP3 | CTGGAGACTCAGAGTCAGTCCCAGACCCTCAACCTCTCGGAGAACCACCAGCAGCCCCCA | 60 |
| AAP4 | CTGGAGCAGGCG--------ACGGACCCCCTGAGGGATCAACTT----CCGGAGC----- | 43 |
| AAP5 | CTGGACC--------CAG---C-GGATCCCAGCAGCTGCAAATCCCAGCCCAACCAGCCT | 48 |
| AAP6 | CTGGCGACTCAGAGTCAGTCCCCGACCCACAACCTCTCGGAGAACCTCCAGCAACCCCCG | 60 |
| AAP7 | CTGGCGACTCAGAGTCAGTCCCCGACCCTCAACCTCTCGGAGAACCTCCAGCAGCGCCCT | 60 |
| AAP8 | CTGGCGACTCAGAGTCAGTTCCAGACCCTCAACCTCTCGGAGAACCTCCAGCAGCGCCCT | 60 |
| AAP9 | ctggcgacacagagtcagtcccagaccctcaaccaatcggagaacctcccgcagccccct | 60 |
| | ****. . * *: * *:.. : .* : *. * * | |
| AAP1 | CTGCTGTGGGACCTACTACAATGGCTTCAGGCGGTGGCGCACCAATGGCAGACAATAACG | 120 |
| AAP2 | CTGGTCTGGGAACTAATACGATGGCTACAGGCAGTGGCGCACCAATGGCAGACAATAACG | 120 |
| AAP3 | CAAGTTTGGGATCTAATACAATGGCTTCAGGCGGTGGCGCACCAATGGCAGACAATAACG | 120 |
| AAP4 | CATGTCTGATGACAG-TGAGATGCGTGCAGCAGCTGGCGGAGCTGCAGTCGAGGGCGGAC | 102 |
| AAP5 | CAAGTTTGGGAGCTGATACAATGTCTGCGGGAGGTGGCGGCCCATTGGGCGACAATAACC | 108 |
| AAP6 | CTGCTGTGGGACCTACTACAATGGCTTCAGGCGGTGGCGCACCAATGGCAGACAATAACG | 120 |
| AAP7 | CTAGTGTGGGATCTGGTACAGTGGCTGCAGGCGGTGGCGCACCAATGGCAGACAATAACG | 120 |
| AAP8 | CTGGTGTGGGACCTAATACAATGGCTGCAGGCGGTGGCGCACCAATGGCAGACAATAACG | 120 |
| AAP9 | caggtgtgggatctcttacaatggcttcaggtggtggcgcaccagtggcagacaataacg | 120 |
| | *: * **. * *: *....** * *.* . ***** . *: .* .** .. ... | |
| AAP1 | AAGGCGCCGACGGAGTGGGTAATGCCTCAGGAAATTGGCATTGCGATTCCACATGGCTGG | 180 |
| AAP2 | AGGGCGCCGACGGAGTGGGTAATTCCTCGGGAAATTGGCATTGCGATTCCACATGGATGG | 180 |
| AAP3 | AGGGTGCCGATGGAGTGGGTAATTCCTCAGGAAATTGGCATTGCGATTCCCAATGGCTGG | 180 |
| AAP4 | AAGGTGCCGATGGAGTGGGTAATGCCTCGGGTGATTGGCATTGCGATTCCACCTGGTCTG | 162 |
| AAP5 | AAGGTGCCGATGGAGTGGGCAATGCCTCGGGAGATTGGCATTGCGATTCCACGTGGATGG | 168 |
| AAP6 | AAGGCGCCGACGGAGTGGGTAATGCCTCAGGAAATTGGCATTGCGATTCCACATGGCTGG | 180 |
| AAP7 | AAGGTGCCGACGGAGTGGGTAATGCCTCAGGAAATTGGCATTGCGATTCCACATGGCTGG | 180 |
| AAP8 | AAGGCGCCGACGGAGTGGGTAGTTCCTCGGGAAATTGGCATTGCGATTCCACATGGCTGG | 180 |
| AAP9 | aaggtgccgatggagtgggtagttcctcgggaaattggcattgcgattcccaatggctgg | 180 |
| | *.** ***** ******** *.* ****.**:.*****************.. *** * | |
| AAP1 | GCGACAGAGTCATCACCACCAGCACCCGCACCTGGGCCTTGCCCACCTACAATAACCACC | 240 |
| AAP2 | GCGACAGAGTCATCACCACCAGCACCCGAACCTGGGCCCTGCCCACCTACAACAACCACC | 240 |
| AAP3 | GCGACAGAGTCATCACCACCAGCACCAGAACCTGGGCCCTGCCCACTTACAACAACCATC | 240 |
| AAP4 | AGGGCCACGTCACGACCACCAGCACCAGAACCTGGGTCTTGCCCACCTACAACAACCACC | 222 |
| AAP5 | GGGACAGAGTCGTCACCAAGTCCACCCGAACCTGGGTGCTGCCCAGCTACAACAACCACC | 228 |
| AAP6 | GCGACAGAGTCATCACCACCAGCACCCGAACATGGGCCTTGCCCACCTATAACAACCACC | 240 |
| AAP7 | GCGACAGAGTCATTACCACCAGCACCCGAACCTGGGCCCTGCCCACCTACAACAACCACC | 240 |
| AAP8 | GCGACAGAGTCATCACCACCAGCACCCGAACCTGGGCCCTGCCCACCTACAACAACCACC | 240 |
| AAP9 | gggacagagtcatcaccaccagcacccgaacctgggccctgcccacctacaacaatcacc | 240 |
| | . *.*...***. ****. : ****.*.**.**** ****** ** ** ** ** * | |
| AAP1 | TCTACAAGCAAATCTCCAGTGCTTCAACGG---GGGCCAGCAACGACAACCACTACTTCG | 297 |
| AAP2 | TCTACAAACAAATTTCCAGCCAATCAGGAGC---C---TCGAACGACAATCACTACTTTG | 294 |
| AAP3 | TCTACAAGCAAATCTCCAGCCAATCA------GGAGCTTCAAACGACAACCACTACTTTG | 294 |
| AAP4 | TCTACAAGCGACTC-----------GG-AGA---GAGCCTGCAGTCCAACACCTACAACG | 267 |
| AAP5 | AGTACCGAGAGATCAAAAGCGGCTCCGTCGA---CGGAAGCAACGCCAACGCCTACTTTG | 285 |
| AAP6 | TCTACAAGCAAATCTCCAGTGCTTCAACGG---GGGCCAGCAACGACAACCACTACTTCG | 297 |
| AAP7 | TCTACAAGCAAATCTCCAGTGAAACTGCAGGT---AGTACCAACGACAACACCTACTTCG | 297 |
| AAP8 | TCTACAAGCAAATCTCCAACGGGACATCGGGAGGAGCCACCAACGACAACACCTACTTCG | 300 |
| AAP9 | tctacaagcaaatctccaacagcacatctggaggatcttcaaatgacaacgcctacttcg | 300 |
| | : ***... ...* .* .*** .****:: * | |
| AAP1 | GCTACAGCACCCCCTGGGGGTATTTTGATTTCAACAGATTCCACTGCCACTTTTCACCAC | 357 |
| AAP2 | GCTACAGCACCCCTTGGGGGTATTTTGACTTCAACAGATTCCACTGCCACTTTTCACCAC | 354 |
| AAP3 | GCTACAGCACCCCTTGGGGGTATTTTGACTTTAACAGATTCCACTGCCACTTCTCACCAC | 354 |
| AAP4 | GATTCTCCACCCCCTGGGGATACTTTGACTTCAACCGCTTCCACTGCCACTTCTCACCAC | 327 |
| AAPS | GATACAGCACCCCCTGGGGGTACTTTGACTTTAACCGCTTCCACAGCCACTGGAGCCCCC | 345 |
| AAP6 | GCTACAGCACCCCCTGGGGGTATTTTGATTTCAACAGATTCCACTGCCATTTCTCACCAC | 357 |
| AAP7 | GCTACAGCACCCCCTGGGGGTATTTTGACTTTAACAGATTCCACTGCCACTTCTCACCAC | 357 |
| AAP8 | GCTACAGCACCCCCTGGGGGTATTTTGACTTTAACAGATTCCACTGCCACTTTTCACCAC | 360 |
| AAP9 | gctacagcaccccctgggggtattttgacttcaacagattccactgccacttctcaccac | 360 |
| | *.*:*: ****** *****.** ***** ** ***.*.******:**** * : .**.* | |
| AAP1 | GTGACTGGCAGCGACTCATCAACAACAATTGGGGATTCCGGCCCAAGAGACTCAACTTCA | 417 |
| AAP2 | GTGACTGGCAAAGACTCATCAACAACAACTGGGGATTCCGACCCAAGAGACTCAACTTCA | 414 |
| AAP3 | GTGACTGGCAGCGACTCATTAACAACAACTGGGGATTCCGGCCCAAGAAACTCAGCTTCA | 414 |
| AAP4 | GTGACTGGCAGCGACTCATCAACAACAACTGGGGCATGCGACCCAAAGCCATGCGGGTCA | 387 |
| AAP5 | GAGACTGGCAAAGACTCATCAACAACTACTGGGGCTTCAGACCCCGGTCCCTCAGAGTCA | 405 |
| AAP6 | GTGACTGGCAGCGACTCATCAACAACAATTGGGGATTCCGGCCCAAGAGACTCAACTTCA | 417 |
| AAP7 | GTGACTGGCAGCGACTCATCAACAACAACTGGGGATTCCGGCCCAAGAAGCTGCGGTTCA | 417 |
| AAP8 | GTGACTGGCAGCGACTCATCAACAACAACTGGGGATTCCGGCCCAAGAGACTCAGCTTCA | 420 |
| AAP9 | gtgactggcagcgactcatcaacaacaactggggattccggcctaagcgactcaacttca | 420 |
| | *:********..******* ******:* *****.:* .*.** ... .* .. *** | |
| AAP1 | AACTCTTCAACATCCAAGTCAAGGAGGTCACGACGAATGATGGCGTCACAACCATCGCTA | 477 |
| AAP2 | AGCTCTTTAACATTCAAGTCAAAGAGGTCACGCAGAATGACGGTACGACGACGATTGCCA | 474 |
| AAP3 | AGCTCTTCAACATCCAAGTTAGAGGGGTCACGCAGAACGATGGCACGACGACTATTGCCA | 474 |
| AAP4 | AAATCTTCAACATCCAGGTCAAGGAGGTCACGACGTCGAACGGCGAGACAACGGTGGCTA | 447 |
| A1P5 | AAATCTTCAACATTCAAGTCAAAGAGGTCACGGTGCAGGACTCCACCACCACCATCGCCA | 465 |
| AAP6 | AGCTCTTCAACATCCAAGTCAAGGAGGTCACGACGAATGATGGCGTCACGACCATCGCTA | 477 |
| AAP7 | AGCTCTTCAACATCCAGGTCAAGGAGGTCACGACGAATGACGGCGTTACGACCATCGCTA | 477 |
| AAP8 | AGCTCTTCAACATCCAGGTCAAGGAGGTCACGCAGAATGAAGGCACCAAGACCATCGCCA | 480 |
| AAP9 | agctcttcaacattcaggtcaaagaggttacggacaacaatggagtcaagaccatcgcca | 480 |
| | *..**** ***** **.** *..*.*** *** . .* . *. ** .* ** * | |
| AAP1 | ATAACCTTACCAGCACGGTTCAAGTCTTCTCGGACTCGGAGTACCAGCTTCCGTACGTCC | 537 |
| AAP2 | ATAACCTTACCAGCACGGTTCAGGTGTTTACTGACTCGGAGTACCAGCTCCCGTACGTCC | 534 |
| AAP3 | ATAACCTTACCAGCACGGTTCAAGTGTTTACGGACTCGGAGTATCAGCTCCCGTACGTGC | 534 |
| AAP4 | ATAACCTTACCAGCACGGTTCAGATCTTTGCGGACTCGTCGTACGAACTGCCGTACGTGA | 507 |
| AAP5 | ACAACCTCACCTCCACCGTCCAAGTGTTTACGGACGACGACTACCAGCTGCCCTACGTCG | 525 |
| AAP6 | ATAACCTTACCAGCACGGTTCAAGTCTTCTCGGACTCGGAGTACCAGTTGCCGTACGTCC | 537 |
| AAP7 | ATAACCTTACCAGCACGATTCAGGTATTCTCGGACTCGGAATACCAGCTGCCGTACGTCC | 537 |
| AAP8 | ATAACCTCACCAGCACCATCCAGGTGTTTACGGACTCGGAGTACCAGCTGCCGTACGTTC | 540 |
| AAP9 | ataaccttaccagcacggtccaggtcttcacggactcagactatcagctcccgtacgtgc | 540 |
| | * ***** ***: *** .* **..* ** * *** . . ** *. * ** ***** | |
| AAP1 | TCGGCTCTGCGCACCAGGGCTGCCTCCCTCCGTTCCCGGCGGACGTGTTCATGA------ | 591 |
| AAP2 | TCGGCTCGGCGCATCAAGGATGCCTCCCGCCGTTCCCAGCAGACGTCTTCATGGTGCCAC | 594 |
| AAP3 | TCGGGTCGGCGCACCAAGGCTGTCTCCCGCCGTTTCCAGCGGACGTCTTCATGGTCCCTC | 594 |
| AAP4 | ------------------------------------------------------------ | 507 |
| AAP5 | TCGGCAACGGGACCGAGGGATGCCTGCCGGCCTTCCCTCCGCAGGTCTTTACGCTGCCGC | 585 |
| AAP6 | TCGGCTCTGCGCACCAGGGCTGCCTCCCTCCGTTCCCGGCGGACGTGTTCATGA------ | 591 |
| AAP7 | TCGGCTCTGCGCACCAGGGCTGCCTGCCTCCGTTCCCGGCGGACGTCTTCATGATTCCTC | 597 |
| AAP8 | TCGGCTCTGCCCACCAGGGCTGCCTGCCTCCGTTCCCGGCGGACGTGTTCATGA------ | 594 |
| AAP9 | tcgggtcggctcacgagggctgcctcccgccgttcccagcggacgttttcatga------ | 594 |
| AAP1 | --------------------- 591 (SEQ ID NO: 32) | |
| AAP2 | AGTATGGATACCTCACCCTGA 615 (SEQ ID NO: 33) | |
| AAP3 | AGTATGGATACCTCACCCTGA 615 (SEQ ID NO: 34) | |
| AAP4 | --------------------- 507 (SEQ ID NO: 35) | |
| AAP5 | AGTACGGTTACGCGACGCTGA 606 (SEQ ID NO: 36) | |
| AAP6 | --------------------- 591 (SEQ ID NO: 37) | |
| AAP7 | AGTACGGCTACCTGA------ 612 (SEQ ID NO: 38) | |
| AAP8 | --------------------- 594 (SEQ ID NO: 39) | |
| AAP9 | --------------------- 594 (SEQ ID NO: 40) | |

1. An engineered assembly activating protein (AAP) comprising components: A, B, and C, wherein
A can be an N terminal domain having the amino acid sequence MENLQQPPLLWDLLQWLQAVAHQWQTITKAPTEWVMPQEIGIAIPHGWATESS (SEQ ID NO:1); or
A can be AAV capsid protein binding domain such as an antibody fragments or binding peptide identified, for example, through phage display;
B can be a linker amino acid sequence which can be from about 10 amino acids to about 240 amino acids in length and can comprise:
MVSKGEELFTGVVPILVELDGDVNGHKFSVSGEGEGDATYGKLTLKFICTTGKLPVPWP TLVTTLTYGVQCFSRYPDHMKQHDFFKSAMPEGYVQERTIFFKDDGNYKTRAEVKFEG DTLVNRIELKGIDFKEDGNILGHKLEYNYNSHNVYIMADKQKNGIKVNFKIRHNIEDGSV QLADHYQQNTPIGDGPVLLPDNHYLSTQSALSKDPNEKRDHMVLLEFVTAAGITLGMD ELYK (SEQ ID NO:2), GAGAGAGQGAGAGAGQGAAAGAGAGAGQT (SEQ ID NO:3), GAGAGQGAGAGAGQGAAAGAGAGAGQGAGAGAGQGAGAGAGQGAAGAGAGAGQT (SEQ ID NO:4), PTPVTAIGPPTTAIQEPPSRIVPTPTSPAIAPPTETMAPPVRDPVPGKPTVTIRTRGAIIQTPT LGPIQPTRVSEAGTTVPGQIRPTLTIPGYVEPTAVITPPTTTTKKPRVSTPKPATPSTDSSTT TTRRPTKKPRTPRPVPRVTTK (SEQ ID NO:5), TLTIPGYVEPTAVITPPTTTTKKPRVSTPKPATPSTDSSTTTTRRPTKKPRTPRPVPRVTT (SEQ ID NO:6), GSSGVRLWATRQAMLGQVHEVPEGWLIFVAEQEELYVRVQNGFRKVQLEARTPLPR (SEQ ID NO:7), and/or AEIEQAKKEIAYLIKKAKEEILEEIKKAKQEIA (SEQ ID NO:8); or
B can comprise a dimerizable domain, a SpyTag system, an FKBP-based system, a leucine zipper system, an immunoglobulin domain, an intein-based system, a protein domain with secondary structure that can comprise alpha-helical, beta strands, coiled coils, proline helix, beta barrel domains and/or other scaffold domains; or
B can comprise a functional domain from other viral or bacterial scaffold proteins that aid in capsid assembly, a bacteriophage Protein B or Protein B domain, a phi 29 connector or scaffolding protein, a SPP1 neck protein, or any combination thereof; and
C can be a C terminal domain having the amino acid sequence KSRRSRRMMASQPSLITLPARFKSSRTRSTSFRTSSA (SEQ ID NO:9); or
C can be an exogenous nuclear/nucleolar localization domain (NLS/NoLS), which can optionally be
(Rpp29)
(SEQ ID NO: 10)
RHKRKEKKKKAKGLSARQRRELR,
(AP3D1)
(SEQ ID NO: 11)
RRHRQKLEKDKRRKKRKEKEERTKGKKKSKK.
(SV40)
(SEQ ID NO: 12)
KRTADGSEFESPKKKRKVE,
and/or
(HIV Rev)
(SEQ ID NO: 13)
RQARRNRRRRWRERQR.

2. The engineered AAP protein of claim 1, wherein the entire T/S rich region (T/S) having the amino acid sequence KSPVLQRGPATTTTTSATAPPGGILISTDSTATFHHVTGSDSSTTIGDSGPRDSTSNS (SEQ ID NO:14) or corresponding T/S rich region in a different AAV serotype is deleted.

3. The engineered AAP protein of claim 1, comprising the proline rich region of the AAP of any of AAV serotypes 1-9 or of the AAP of an AAV rhesus monkey isolate, optionally PPAPAPGPCPP (SEQ ID NO:15) of AAV1; or PPAPEPGPCPP (SEQ ID NO:16) of AAV2.

4. The engineered AAP of any of claim 1, wherein the presence of the linker amino acid sequence in the engineered AAP imparts increased stability, improved ability to support viral capsid assembly, nucleolar transport activity, nuclear transport activity, ability to be detected, ability to bind other proteins, ability to bind other nucleic acid molecules, ability to binds other macromolecules, ability to form multimers in the presences or absence of other co-factors, ability to increase virus particle yield in a different production system, and any combination thereof, to the engineered AAP relative to an AAP without the linker amino acid sequence.

5. The engineered AAP of claim 1, wherein the AAP is from an adeno-associated virus (AAV).

6. A producer cell line for production of AAV particles, comprising a heterologous nucleotide sequence encoding the engineered AAP of claim 1.

7. The producer cell line of claim 6, wherein the cell line is a mammalian cell line, an insect cell line, a yeast cell line, a protozoan cell line or a bacterial cell line.

8. The producer cell line of claim 6, wherein the heterologous nucleotide sequence is integrated into the genome of the cells of the producer cell line.

9. The producer cell line of claim 6, wherein the heterologous nucleotide sequence is transiently present in the cells of the producer cell line.

10. The producer cell line of claim 6, further comprising regulatory elements to control expression of the heterologous nucleotide sequence.

11. The producer cell line of claim 10, comprising regulatory elements for genetic control; for epigenetic control; for transcriptional control; for post-transcriptional control; for translational control; for post-translational control, and any combination thereof.
